SEQUENCE LISTING (54 sequences)

SEQ ID NO: 1 - protein, 215 aa, Artificial Sequence
  mAb CS-D7 light chain
EIVMTQSPATLSVSPGERATLSCRASQYVSDNLAWYQQKPGQAPRLLIYGASTRATGVPARFSGSGSGTEFTLTISSLQSEDFAVYYCQQYNNWRPVTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 456 aa, Artificial Sequence
  mAb CS-D7 heavy chain
QVQLQESGPGLVKPSETLSLTCTVSGGSIRSSSYYWGWFRQTPGKGLEWLGNVFFSGSAYYNPSLKNRVTISIDTSENQSSLKLTSVTAADTAVYYCARPQAYSHDSSGHSPFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 112 aa, Artificial Sequence
  mAb CS-E11 light chain variable region
QAVLTQPSSVSGAPGQRVTISCTGDRSNIGATYDVHWYQQLPGRAPKLLIYGNHNRPSGVPERFSGSKSGSSASLAIAGLQAEDEADYYCQSYDSGLSGYVFGTGTKVTVLG

SEQ ID NO: 4 - protein, 126 aa, Artificial Sequence
  mAb CS-E11 heavy chain variable region
QVQLQESGPGLVKPSETLSLTCTVSGGSIRSSSYYWGWFRQTPGKGLEWLGNVFFSGSAYYNPSLKNRVTISIDTSENQSSLKLTSVTAADTAVYYCARPQAYSHDSSGHSPFDLWGQGTLVTVSS

SEQ ID NO: 5 - protein, 113 aa, Artificial Sequence
  mAb CS-C10 light chain variable region
QAVLTQPSSVSGAPGQRVTISCTGGSSNIGAGYDVHWYQQIPGTAPKLLIYGNSNRPSGVPDRFSGSKSGTSASLAITGLQAEDEADYYCQSYDSSLNGPVVFGGGTKVTVLG

SEQ ID NO: 6 - protein, 126 aa, Artificial Sequence
  mAb CS-C10 heavy chain variable region
QVQLQESGPGLVKPSETLSLTCTVSGGSIRSSSYYWGWFRQTPGKGLEWLGNVFFSGSAYYNPSLKSRVTISIDTSENQSSLKLTSVTAADTAVYYCARPQAYSHDSSGHSPFDLWGRGTMVTVSS

SEQ ID NO: 7 - protein, 108 aa, Artificial Sequence
  mAb CS-A10 light chain variable region
QSVLTQPPSVSVSPGQTATITCSGDNLGDKSVSWYQQKAGQSPVLVMSQGSKRPLGIPDRISGSNSGTTATLTISGVQTVDEADFYCQTWDRYTGVVFGGGTKVTVLG

SEQ ID NO: 8 - protein, 126 aa, Artificial Sequence
  mAb CS-A10 heavy chain variable region
RVQLQESGPGLVKPSGTLSLTCTVSGGSIRSSSYYWGWFRQTPGKGLEWLGNVFFSGSAYYNPSLKGRVTISIDTSENQSSLKLTSVTAADTAVYYCARPQAYSHDSSGHSPFDLWGQGTLVTVSS

SEQ ID NO: 9 - protein, 111 aa, Artificial Sequence
  mAb BMV-H11 light chain variable region
QSVLTQPASVSGSPGQSITISCTGTSSDVGGYNYVSWYQQHPGKAPKLMIYEGSKRPSGVSNRFSGSKSGNTASLTISGLQAEDEADYYCSSYTTRSTRVFGGGTKLTVLG

SEQ ID NO: 10 - protein, 128 aa, Artificial Sequence
  mAb BMV-H11 heavy chain variable region
RVQLQESGPGLVKPSETLSLICTVSGGSISSSSYYWGWIRQPPGKGLEWIGNMFYSGGAYYNPSLKSRVSISVGPSSNQFSLKLSSVTAADTAVYYCARPLGYNFDSSGQGKSAFEIWGQGTLVTVSS

SEQ ID NO: 11 - protein, 111 aa, Artificial Sequence; identical to SEQ ID NO: 9
  mAb BMV-E6 light chain variable region

SEQ ID NO: 12 - protein, 128 aa, Artificial Sequence
  mAb BMV-E6 heavy chain variable region
QVQLQESGPGLVKPSETLSLTCTVSGGSISSSSYYWGWIRQPPGKGLEWIGNMFYSGSAYYNPSLKSRVSISVGPSSNQFSLKLTSVTAADTAVYYCARPLGHNFDSSGQGEGAFEIWGRGTLVTVSS

SEQ ID NO: 13 - protein, 111 aa, Artificial Sequence; identical to SEQ ID NO: 9
  mAb BMV-D4 light chain variable region

SEQ ID NO: 14 - protein, 128 aa, Artificial Sequence
  mAb BMV-D4 heavy chain variable region
QVQLQESGPGLVKPSETLSLTCTVSGGSISSSSYYWGWIRQPPGKGLEWIGNMFYSGGAYYNPSLKNRVSISVGPSSNQFSLKLSSVTAADTAVYYCARPLGYNFDSSGQGKSAFEIWGKGTMVTVSS

SEQ ID NO: 15 - protein, 111 aa, Artificial Sequence
  mAb BMV-C2 light chain variable region
QSVLTQPPSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLG

SEQ ID NO: 16 - protein, 128 aa, Artificial Sequence
  mAb BMV-C2 heavy chain variable region
EVQLQESGPGLVKPSETLSLTCTVSGGSISSSSYYWGWIRQPPGKGLEWIGNMFYSGSAYYNPSLKSRVSISVGPSSNQFSLKLTSVTAADTAVYYCARPLGHNFDSSGQGEGAFEIWGQGTLVTVSS

SEQ ID NO: 17 - protein, 11 aa, Artificial Sequence
  mAb CS-D7 VL CDR1
RASQYVSDNLA

SEQ ID NO: 18 - protein, 7 aa, Artificial Sequence
  mAb CS-D7 VL CDR2
GASTRAT

SEQ ID NO: 19 - protein, 10 aa, Artificial Sequence
  mAb CS-D7 VL CDR3
QQYNNWRPVT

SEQ ID NO: 20 - protein, 14 aa, Artificial Sequence
  mAb CS-E11 VL CDR1
TGDRSNIGATYDVH

SEQ ID NO: 21 - protein, 7 aa, Artificial Sequence
  mAb CS-E11 VL CDR2
GNHNRPS

SEQ ID NO: 22 - protein, 11 aa, Artificial Sequence
  mAb CS-E11 VL CDR3
QSYDSGLSGYV

SEQ ID NO: 23 - protein, 14 aa, Artificial Sequence
  mAb CS-D10 VL CDR1
TGGSSNIGAGYDVH

SEQ ID NO: 24 - protein, 7 aa, Artificial Sequence
  mAb CS-D10 VL CDR2
GNSNRPS

SEQ ID NO: 25 - protein, 12 aa, Artificial Sequence
  mAb CS-D10 VL CDR3
QSYDSSLNGPVV

SEQ ID NO: 26 - protein, 11 aa, Artificial Sequence
  mAb CS-A10 VL CDR1
SGDNLGDKSVS

SEQ ID NO: 27 - protein, 7 aa, Artificial Sequence
  mAb CS-A10 VL CDR2
QGSKRPL

SEQ ID NO: 28 - protein, 10 aa, Artificial Sequence
  mAb CS-A10 VL CDR3
QTWDRYTGVV

SEQ ID NO: 29 - protein, 14 aa, Artificial Sequence
  mAbs BMV-H11, BMV-E6 and BMV-D4 VL CDR1
TGTSSDVGGYNYVS

SEQ ID NO: 30 - protein, 7 aa, Artificial Sequence
  mAbs BMV-H11, BMV-E6 and BMV-D4 VL CDR2
EGSKRPS

SEQ ID NO: 31 - protein, 10 aa, Artificial Sequence
  mAbs BMV-H11, BMV-E6 and BMV-D4 VL CDR3
SSYTTRSTRV

SEQ ID NO: 32 - protein, 13 aa, Artificial Sequence
  mAb BMV-C2 VL CDR1
SGSTSNIGNNYVS

SEQ ID NO: 33 - protein, 7 aa, Artificial Sequence
  mAb BMV-C2 VL CDR2
DVSKRPS

SEQ ID NO: 34 - protein, 11 aa, Artificial Sequence
  mAb BMV-C2 VL CDR3
AAWDDSLSEFL

SEQ ID NO: 35 - protein, 12 aa, Artificial Sequence
  mAbs CS-D7, CS-E11, CS-D10 and CS-A10 VH CDR1
GGSIRSSSYYWG

SEQ ID NO: 36 - protein, 16 aa, Artificial Sequence
  mAbs CS-D7 and CS-E11  VH CDR2
NVFFSGSAYYNPSLKN

SEQ ID NO: 37 - protein, 16 aa, Artificial Sequence
  mAbs CS-D7, CS-E11, CS-D10 and CS-A10  VH CDR3
PQAYSHDSSGHSPFDL

SEQ ID NO: 38 - protein, 16 aa, Artificial Sequence
  mAb CS-D10 VH CDR2
NVFFSGSAYYNPSLKS

SEQ ID NO: 39 - protein, 16 aa, Artificial Sequence
  mAb CS-A10 VH CDR2
NVFFSGSAYYNPSLKG

SEQ ID NO: 40 - protein, 12 aa, Artificial Sequence
  mAbs BMV-H11, BMV-E6, BMV-D4 and BMV-C2 VH CDR1
GGSISSSSYYWG

SEQ ID NO: 41 - protein, 16 aa, Artificial Sequence
  mAb BMV-H11 VH CDR2
NMFYSGGAYYNPSLKS

SEQ ID NO: 42 - protein, 18 aa, Artificial Sequence
  mAbs BMV-H11 and BMV-C2 VH CDR3
PLGYNFDSSGQGKSAFEI

SEQ ID NO: 43 - protein, 16 aa, Artificial Sequence
  mAbs BMV-E6 and BMV-C2 VH CDR2
NMFYSGSAYYNPSLKS

SEQ ID NO: 44 - protein, 16 aa, Artificial Sequence
  mAb BMV-D4 VH CDR2
NMFYSGGAYYNPSLKN

SEQ ID NO: 45 - protein, 18 aa, Artificial Sequence
  mAbs BMV-E6 and BMV-C2 VH CDR3
PLGHNFDSSGQGEGAFEI

SEQ ID NO: 46 - protein, 12 aa, Artificial Sequence
  consensus sequence of SEQ ID NOs 35 and 40
GGSIXSSSYYWG

SEQ ID NO: 47 - protein, 645 aa, Staphylococcus aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 48 - protein, 105 aa, Homo sapien
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 49 - protein, 19 aa, Artificial Sequence
  heavy chain leader
MEWSWVFLFFLSVTTGVHS

SEQ ID NO: 50 - protein, 20 aa, Artificial Sequence
  light chain leader
MSVPTQVLGLLLLWLTDARC

SEQ ID NO: 51 - DNA, 36 bp, Artificial Sequence
  oligonucleotide primer
acagatgccagatgcgaaattgtgatgacacagtct

SEQ ID NO: 52 - DNA, 36 bp, Artificial Sequence
  oligonucleotide primer
tgcagccaccgtacgtttaatctccagtcgtgtccc

SEQ ID NO: 53 - DNA, 36 bp, Artificial Sequence
  oligonucleotide primer
acaggtgtccactcgcaggtgcagctgcaggagtcg

SEQ ID NO: 54 - DNA, 36 bp, Artificial Sequence
  oligonucleotide primer
gcccttggtggatgcactcgagacggtgaccagggt

(19) United States
(12) Patent Application Publication
(10) Pub. No.: US 2010/0166772 A1
(43) Pub. Date: Jul. 1, 2010

(54) ANTIGEN-BINDING PROTEINS TARGETING S. AUREUS ORF0657n

(76) Inventors: Annaliesa S. Anderson, Upper Saddle River, NJ (US); Desmond J. Clark, Wyncote, PA (US); Zhiqiang An, Ambler, PA (US); Fubao Wang, Dresher, PA (US); Susan L. Secore, Telford, PA (US); Eberhard Durr, Quakertown, PA (US); Leslie D. Cope, Hatfield, PA (US); Tessie McNeely, Gwynedd Valley, PA (US)

Correspondence Address: MERCK, P O BOX 2000, RAHWAY, NJ, 07065-0907, US

(21) Appl. No.: 12/601,933

(22) PCT Filed: May 29, 2008
(86) PCT No.: PCT/US08/06791

§ 371 Date: Nov. 25, 2009

Related U.S. Application Data
(60) Provisional application No. 61/007,998, filed on Dec. 17, 2007.
(60) Provisional application No. 60/932,788, filed on May 31, 2007.

(51) Int. Cl. A61K 39/40; C07K 16/12; C07H 21/04; C12N 5/10; C12P 21/06; A61K 39/395; C07K 14/00

(52) U.S. Cl. 424/165.1; 530/387.1; 536/23.53; 435/325; 435/69.6; 424/130.1; 530/350

(57) ABSTRACT

The present invention features antigen binding proteins that bind to a region found to have an epitope that can be targeted to provide protection against S. aureus infection. The region is designated herein as the “CS-D7” target region. The CS-D7 target region provides an S. aureus ORF0657n epitope that can be targeted to reduce the likelihood or severity of an S. aureus infection.

### CROSS-REFERENCE TO RELATED APPLICATIONS

The present application claims the benefit of U.S. Provisional Application No. 60/932,788, filed May 31, 2007, and U.S. Provisional Application No. 61/007,998, filed Dec. 17, 2007, hereby incorporated by reference herein.

### BACKGROUND OF THE INVENTION

The references cited throughout the present application are not admitted to be prior art to the claimed invention.

Staphylococcus aureus (S. aureus) is a pathogen responsible for a wide range of diseases and conditions. Examples of diseases and conditions caused by S. aureus include bacteremia, infective endocarditis, folliculitis, furuncle, carbuncle, impetigo, bullous impetigo, cellulitis, botryomyosis, toxic shock syndrome, scalded skin syndrome, central nervous system infections, infective and inflammatory eye disease, osteomyelitis and other infections of joints and bones, and respiratory tract infections. (The Staphylococci in Human Disease, Crossley and Archer (eds.), Churchill Livingstone Inc. 1997.)

Immunological-based strategies can be employed to control S. aureus infections and the spread of S. aureus. Immunological-based strategies include passive and active immunization. Passive immunization employs immunoglobulins targeting S. aureus. Active immunization induces immune responses against S. aureus.

### SUMMARY OF THE INVENTION

The present invention features antigen binding proteins that bind to a region found to have an epitope that can be targeted to provide protection against S. aureus infection. The region is designated herein as the “CS-D7” target region. The CS-D7 target region provides an S. aureus ORF0657n epitope that can be targeted to reduce the likelihood or severity of an S. aureus infection.

Thus, a first aspect of the present invention features an isolated antigen binding protein comprising a first variable region and a second variable region, wherein the variable regions bind to a CS-D7 target region. The CS-D7 target region is specifically targeted by monoclonal antibody CS-D7 (mAb CS-D7). MAb CS-D7 is an immunoglobulin having two light chains with an amino acid sequence of SEQ ID NO: 1 and two heavy chains with an amino acid sequence of SEQ ID NO: 2.

Reference to “isolated” indicates a different form than found in nature. The different form can be, for example, a different purity than found in nature and/or a structure that is not found in nature. A structure not found in nature includes recombinant structures where different regions are combined together, for example, humanized antibodies where one or more murine complementarity determining regions is inserted onto a human framework scaffold or a murine antibody is resurfaced to resemble the surface residues of a human antibody, hybrid antibodies where one or more complementarity determining regions from an antigen binding protein is inserted into a different framework scaffold, and antibodies derived from natural human sequences where genes coding for light and heavy variable domains were randomly combined together.

The isolated protein is preferably substantially free of serum proteins. A protein substantially free of serum proteins is present in an environment lacking most or all serum proteins.

A “variable region” has the structure of an antibody variable region from a heavy or light chain. Antibody heavy and light chain variable regions contain three complementarity determining regions interspaced onto a framework. The complementarity determining regions are primarily responsible for recognizing a particular epitope.

A target region is defined with respect to the ORF0657n region (SEQ ID NO: 47) bound by mAb CS-D7. A protein binding the CS-D7 target region reduces binding of mAb CS-D7 to ORF0657n by at least about 20%, preferably at least about 50%, when excess and equal amounts of the competing protein and monoclonal antibody are employed using a Luminex based inhibition assay.

Reference to “protein” indicates a contiguous amino acid sequence and does not provide a minimum or maximum size limitation. One or more amino acids present in the protein may contain a post-translational modification, such as glycosylation or disulfide bond formation.

A preferred antigen binding protein is a monoclonal antibody. Reference to a “monoclonal antibody” indicates a collection of antibodies having the same, or substantially the same, structure. The variation in the antibodies is that which would occur if the antibodies were produced from the same construct(s).

Monoclonal antibodies can be produced, for example, from a particular hybridoma and from a recombinant cell containing one or more recombinant genes encoding the antibody. The antibody may be encoded by more than one recombinant gene where, for example, one gene encodes the heavy chain and one gene encodes the light chain.

Another aspect of the present invention describes a nucleic acid comprising one or more recombinant genes encoding either, or both of, an antigen binding protein Vₕ region or Vₗ region, wherein the antigen binding protein binds to the CS-D7 target region. Multiple recombinant genes are useful, for example, where one gene encodes an antibody heavy chain or fragment thereof containing the Vₕ region and another gene encodes an antibody light chain or fragment thereof containing the Vₗ region.

A recombinant gene contains recombinant nucleic acid encoding a protein along with regulatory elements for proper transcription and processing (which may include translational and post translational elements). The recombinant nucleic acid by virtue of its sequence and/or form does not occur in nature. Examples of recombinant nucleic acid include purified nucleic acid, two or more nucleic acid regions combined together providing a different nucleic acid than found in nature, and the absence of one or more nucleic acid regions (e.g., upstream or downstream regions) that are naturally associated with each other.

Another aspect of the present invention features a recombinant cell comprising one or more recombinant genes encoding either, or both of, an antigen binding protein Vₕ region or Vₗ region. Preferably, the recombinant cell expresses both the Vₕ and Vₗ regions.

Another aspect of the present invention comprises a method of producing a protein comprising an antibody variable region. The method comprises the steps of: (a) growing a recombinant cell comprising recombinant nucleic acid encoding the protein under conditions wherein the protein is expressed; and (b) purifying the protein. Preferably, the protein is a complete antigen binding protein.

Another aspect of the present invention describes a pharmaceutical composition. The composition comprises a therapeutically effective amount of an antigen binding protein described herein and a pharmaceutically acceptable carrier.

A therapeutically effective amount is an amount sufficient to provide a useful therapeutic or prophylactic effect. For a patient infected with S. aureus, an effective amount is sufficient to achieve one or more of the following effects: reduce the ability of S. aureus to propagate in the patient or reduce the amount of S. aureus in the patient. For a patient not infected with S. aureus, an effective amount is sufficient to achieve one or more of the following: a reduced susceptibility to S. aureus infection or a reduced ability of the infecting bacterium to establish persistent infection for chronic disease.

Another aspect of the present invention describes the use of a therapeutically effective amount of an antigen binding protein in the preparation of a medicament for treating (therapeutically or prophylactically) against S. aureus infection.

Another aspect of the present invention features a method of treating a patient against a S. aureus infection. The method comprises the step of administering to the patient an effective amount of an antigen binding protein described herein, including a pharmaceutical composition thereof. The patient being treated may, or may not, be infected with S. aureus. Preferably, the patient is a human.

Another aspect of the present invention features a polypeptide comprising an amino acid sequence with at least a 95% sequence identity to amino acids 42-342 of SEQ ID NO: 47, wherein the polypeptide is up to 350 amino acids in length.

Reference to open-ended terms such as “comprises” allows for additional elements or steps. Occasionally, phrases such as “one or more” are used with or without open-ended terms to highlight the possibility of additional elements or steps.

Unless explicitly stated, reference to terms such as “a” or “an” is not limited to one. For example, “a cell” does not exclude “cells”. Occasionally, phrases such as one or more are used to highlight the possible presence of a plurality.

Other features and advantages of the present invention are apparent from the additional descriptions provided herein, including the different examples. The provided examples illustrate different components and methodology useful in practicing the present invention. The examples do not limit the claimed invention. Based on the present disclosure, the skilled artisan can identify and employ other components and methodology useful for practicing the present invention.

### BRIEF DESCRIPTION OF THE DRAWINGS

FIG. 1 illustrates the structure of an IgG molecule. “Vₗ” refers to a light chain variable region. “Vₕ” refers to a heavy chain variable region. “Cₗ” refers to a light chain constant region. “CH₁”, “CH₂” and “CH₃” are heavy chain constant regions. Dashed lines indicate disulfide bonds.

FIG. 2 provides a sequence comparison of the mAb CS-D7 light chain variable region (amino acids 1-108 of SEQ ID NO: 1), mAb CS-E11 light chain variable region (SEQ ID NO: 3), mAb CS-D10 light chain variable region (SEQ ID NO: 5), mAb CS-A10 light chain variable region (SEQ ID NO: 7), mAb BMV-H11 light chain variable region (SEQ ID NO: 9), mAb BMV-E6 light chain variable region (SEQ ID NO: 11), mAb BMV-D4 light chain variable region (SEQ ID NO: 13), and mAb BMV-C2 light chain variable region (SEQ ID NO: 15). Complementarity determining regions 1, 2 and 3 are shown in bold, with a SEQ ID NO: identifying different CDR sequences.

FIG. 3 provides a sequence comparison of the mAb CS-D7 heavy chain variable region (amino acids 1-126 of SEQ ID NO: 2), mAb CS-E11 heavy chain variable region (SEQ ID NO: 4), mAb CS-D10 heavy chain variable region (SEQ ID NO: 6), mAb CS-A10 heavy chain variable region (SEQ ID NO: 8), mAb BMV-H11 heavy chain variable region (SEQ ID NO: 10), mAb BMV-E6 heavy chain variable region (SEQ ID NO: 12), mAb BMV-D4 heavy chain variable region (SEQ ID NO: 14), and mAb BMV-C2 heavy chain variable region (SEQ ID NO: 16). Complementarity determining regions 1, 2 and 3 are shown in bold with a SEQ ID NO: identifying different CDR sequences.

FIG. 4 illustrates the ability of mAb CS-D7 to provide protection against S. aureus using an opsonophagocytosis activity (OPA) assay.

FIG. 5 illustrates the ability of mAb CS-D10 to provide protection against S. aureus using an opsonophagocytosis activity (OPA) assay.

FIG. 6 illustrates the ability of mAb CS-D7 to reduce S. aureus bacteremia.

### DETAILED DESCRIPTION OF THE INVENTION

Due to their ability to bind the CS-D7 target region, the antigen binding proteins described herein can be used, for example, as a tool in the production, characterization, or study of ORF0657n based antigens; and/or as an agent to treat S. aureus infection. ORF0657n is an S. aureus protein located at the S. aureus outer membrane. ORF0657n has been found to be well conserved in different strains of S. aureus. (Anderson et al., International Publication No. WO 2005/009379, International Publication Date Feb. 3, 2005.) Different ORF0657n derivatives can be used to produce a protective immune response against S. aureus infection. (Anderson et al., International Publication No. WO 2005/009379, International Publication Date Feb. 3, 2005.)

### I. ANTIGEN BINDING PROTEIN

Antigen binding proteins contain an antibody variable region providing for specific binding to an epitope. The antibody variable region can be present in, for example, a complete antibody, an antibody fragment, and a recombinant derivative of an antibody or antibody fragment.

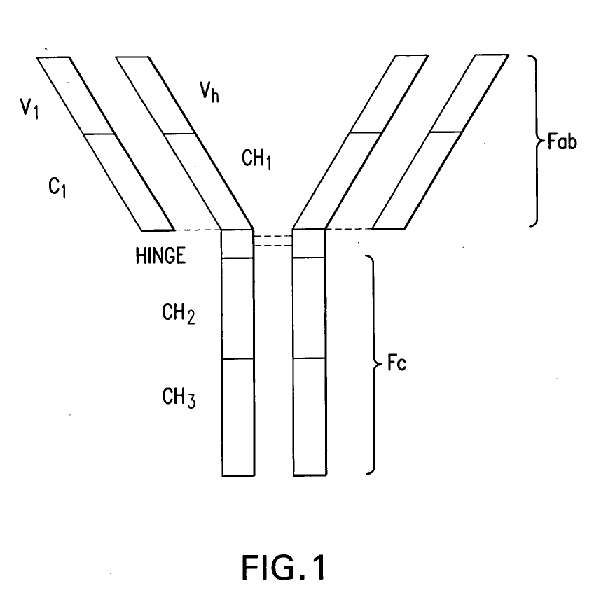

Different classes of antibodies have different structures. Different antibody regions can be illustrated by reference to IgG (FIG. 1). An IgG molecule contains four amino acid chains: two longer length heavy chains and two shorter light chains. The heavy and light chains each contain a constant region and a variable region. Within the variable regions are three hypervariable regions responsible for antigen specificity. (See, for example, Breitling et al., Recombinant Antibodies, John Wiley & Sons, Inc. and Spektrum Akademischer Verlag, 1999; and Lewin, Genes IV, Oxford University Press and Cell Press, 1990.)

The hypervariable regions (also referred to as complementarity determining regions) are interposed between more conserved flanking regions (also referred to as framework regions). Amino acids associated with framework regions and complementarity determining regions (CDRs) can be numbered and aligned as described by Kabat et al., Sequences of Proteins of Immunological Interest, U.S. Department of Health and Human Services, 1991.

The two heavy chain carboxyl regions are constant regions joined by disulfide binding to produce an Fc region. The Fc region is important for providing effector functions. (Presta, Advanced Drug Delivery Reviews 58:640-656, 2006.) Each of the two heavy chains making up the Fc region extend into different Fab regions through a hinge region.

In higher vertebrates there are two classes of light chains and five classes of heavy chains. The light chains are either κ or λ. The heavy chains define the antibody class and are either α, δ, ε, γ, or μ. For example, IgG has a γ heavy chain. Subclasses also exist for different types of heavy chains such as human γ₁, γ₂, γ₃ and γ₄. Heavy chains impart a distinctive conformation to hinge and tail regions. (Lewin, Genes IV, Oxford University Press and Cell Press, 1990.)

Antibody fragments containing an antibody variable region include Fv, Fab and Fab₂ regions. Each Fab region contains a light chain made up of a variable region and a constant region, as well as a heavy chain region containing a variable region and a constant region. A light chain is joined to a heavy chain by disulfide bonding through constant regions. The light and heavy chain variable regions of a Fab region provide for an Fv region that participates in antigen binding.

The antibody variable region can be present in a recombinant derivative. Examples of recombinant derivatives include single-chain antibodies, diabody, triabody, tetrabody, and miniantibody. (Kipriyanov et al, Molecular Biotechnology 26:39-60, 2004.)

The antigen binding protein can contain one or more variable regions recognizing the same or different epitopes. (Kipriyanov et al., Molecular Biotechnology 26:39-60, 2004.)

### II. GENERATION OF ANTIGEN BINDING PROTEINS DIRECTED TO THE CS-D7 TARGET REGION

Antigen binding proteins directed to the CS-D7 target region can be obtained using different techniques, such as those making use of antigen binding proteins that bind to the CS-D7 target region and screening for additional binding proteins that bind the target region. The ability of an antibody to bind the CS-D7 target region can be evaluated using a Luminex assay and mAb CS-D7 (see Example 2 infra). Antigen binding proteins that bind to the CS-D7 target region can be used in different ways for obtaining additional binding proteins, such as using sequence information from the antigen binding protein and/or modifying the antigen binding protein.

### II.A. VARIABLE REGION DESIGN

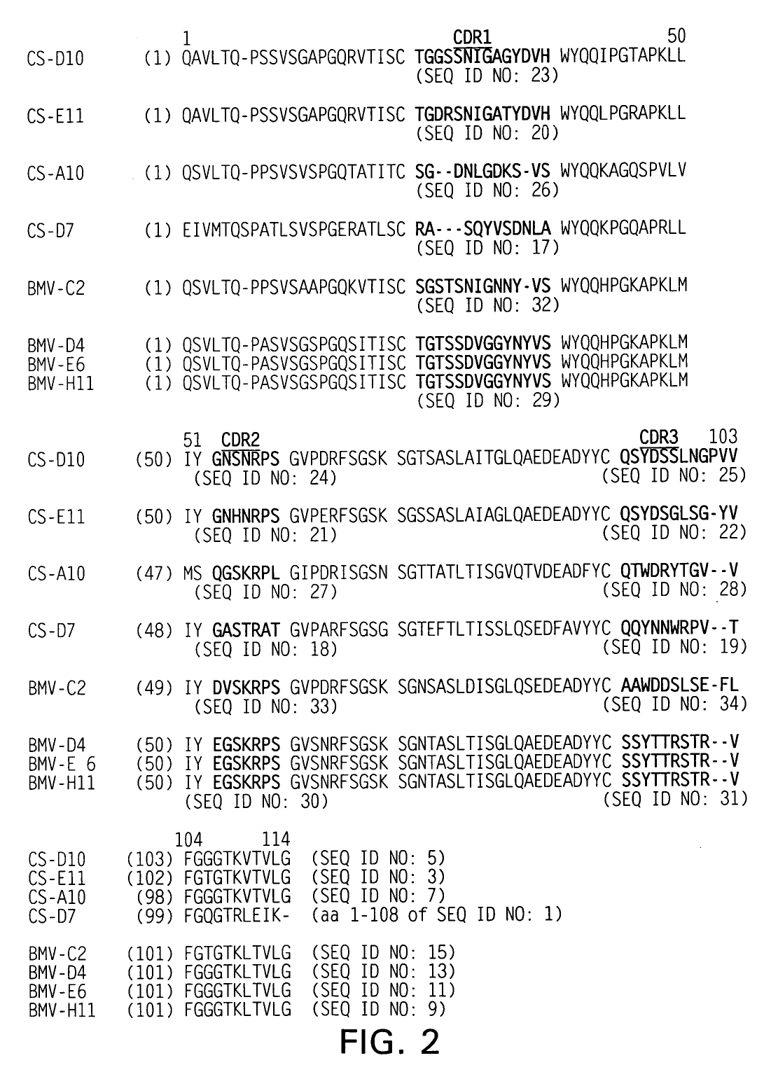

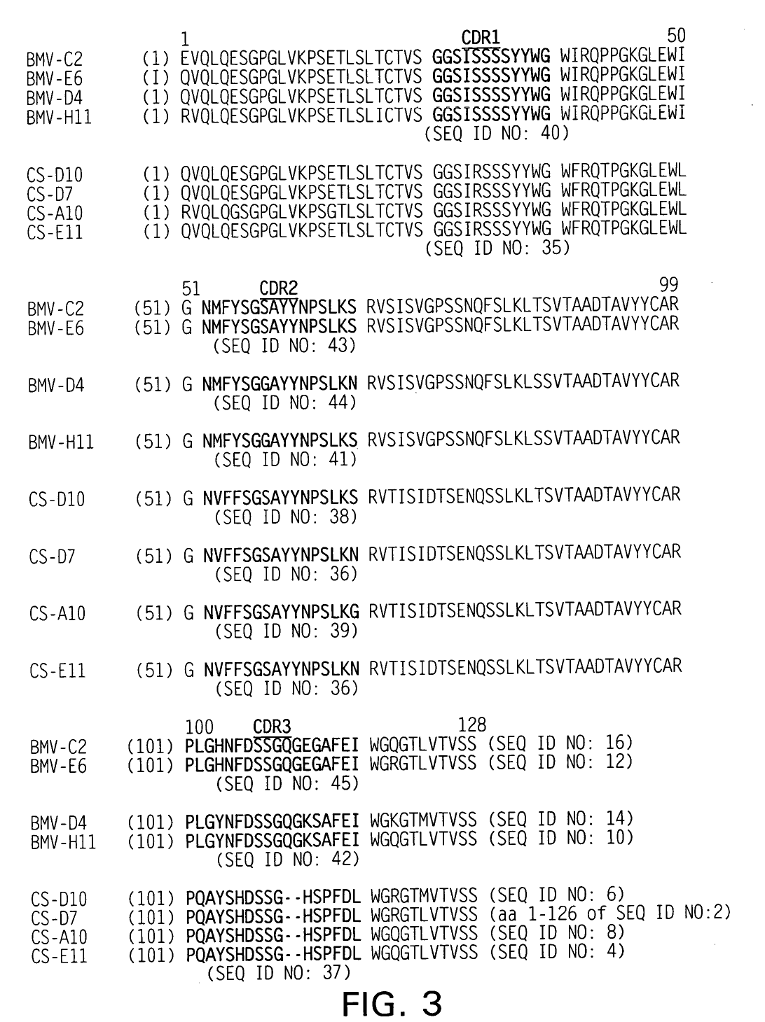

Variable regions for antigen binding proteins can be designed based upon variable regions binding the CS-D7 target region. Based on a Luminex assay, mAbs designated CS-D7, CS-E11, CS-D10, CS-A10, BMV-H11, BMV-E6, BMV-D4, and BMV-C2 were found to bind to the same region. FIG. 2 provides a sequence comparison of the light chain variable regions for these different antibodies. FIG. 3 provides a sequence comparison of the different heavy variable regions for these different antibodies.

The sequence comparisons in FIGS. 2 and 3 provide examples of different variable region CDRs and framework sequences for antigen binding proteins. The antibody variable regions illustrated in FIGS. 2 and 3 were derived from either a peripheral blood lymphocytes library (designated “BMV”) or spleen lymphocytes (designated “CS”).

CDRs are primarily responsible for binding to a particular epitope. Within a particular CDR, there are few specificity determining residues (SDRs) which are of greater importance for binding to an epitope. (Kashmiri et al., Methods 36:25-34, 2005, Presta, Advanced Drug Delivery Reviews 58:640-656, 2006). SDRs can be identified, for example, through the help of antigen-antibody three-dimensional structures and mutational analysis of antibody combining sites. (Kashmiri et al., Methods 36:25-34, 2005.)

The framework regions help provide an overall structure and are more tolerant of different amino acid variations than CDRs. A variety of different naturally occurring framework regions are well-know in the art. (See for example, Kabat et al., Sequences of Proteins of Immunological Interest, U.S. Department of Health and Human Services, 1991.)

The variable regions for mAbs CS-D7, CS-E11, CS-D10, CS-A10, BMV-H11, BMV-E6, BMV-D4, and BMV-C2 and corresponding CDR SEQ ID NOs: are noted in the FIGS. 2 and 3 sequence comparisons. Table 1 provides a summary of the CDR SEQ ID NOs.

TABLE 1

| | Light Chain Variable Region | | | Heavy Chain Variable Region | | |
|---|---|---|---|---|---|---|
| | CDR₁ | CDR₂ | CDR₃ | CDR₁ | CDR₂ | CDR₃ |
| | SEQ ID | SEQ ID | SEQ ID | SEQ ID | SEQ ID | SEQ ID |
| mAb | NO: | NO: | NO: | NO: | NO: | NO: |
| CS-D7 | 17 | 18 | 19 | 35 | 36 | 37 |
| CS-E11 | 20 | 21 | 22 | 35 | 36 | 37 |
| CS-D10 | 23 | 24 | 25 | 35 | 38 | 37 |
| CS-A10 | 26 | 27 | 28 | 35 | 39 | 37 |
| BMV-H11 | 29 | 30 | 31 | 40 | 41 | 42 |
| BMV-E6 | 29 | 30 | 31 | 40 | 43 | 45 |
| BMV-D4 | 29 | 30 | 31 | 40 | 44 | 42 |
| BMV-C2 | 32 | 33 | 34 | 40 | 43 | 45 |

The sequence comparison illustrated in FIGS. 2 and 3 provides examples of different amino acid substitutions within framework and CDR regions. Alterations can be made to both framework regions and CDRs and still retain specificity for the CS-D7 binding region.

### II.B. SCREENING FOR ADDITIONAL BINDING PROTEINS

Additional binding proteins targeting the CS-D7 target region can be obtained using full-length ORF0657n or a polypeptide that provides the epitope recognized by mAb CS-D7. The CS-D7 target region appears to be located within approximately amino acids 42-342 of ORF0657n (SEQ ID NO:47).

A variety of techniques are available to select for a protein recognizing an antigen. Examples of such techniques include the use of phage display technology and hybridoma production. Human antibodies can be produced starting with a human phage display library or using chimeric mice such as a XenoMouse or Trans-Chromo mouse. (E.g., Azzazy et al., Clinical Biochemistry 35:425-445, 2002, Berger et al., Am. J. Med. Sci. 324(1):14-40, 2002.)

Non-human antibodies, such as murine antibodies, can also be obtained. The potential generation of human anti-mouse antibodies can be reduced using techniques such as murine antibody humanization, de-immunization and chimeric antibody production. (See, for example, O'Brien et al., Humanization of Monoclonal Antibodies by CDR Grafting, p 81-100, From Methods in Molecular Biology Vol. 207: Recombinant antibodies for Cancer Therapy: Methods and Protocols (Eds. Welschof and Krauss) Humana Press, Totowa, N.J., 2003; Kipriyanov et al., Molecular Biotechnology 26:39-60, 2004; Gonzales et al., Tumor Biol. 26:31-43, 2005, Presta, Advanced Drug Delivery Reviews 58:640-656, 2006, Tsurushita et al., Methods 36:69-83, 2005, Roque et al., Biotechnol. Prog. 20:639-654, 2004.)

Techniques such as affinity maturation can be used to further enhance the ability of an antigen binding protein to selectively bind to a target. Affinity maturation can be performed, for example, by introducing mutations into a CDR region and determining the effect of the mutations on binding. Different techniques may be employed to introduce the mutations. (Rajpal et al., PNAS 102:8466-8471, 2005, Presta, Advanced Drug Delivery Reviews 58:640-656, 2006.)

### II.C. ADDITIONAL COMPONENTS

Antigen binding proteins may contain additional components including, but not limited to, components other than variable regions or additional variable regions that provide, or help provide, useful activities. Useful activities include antibody effector functions such as antibody-dependent cellular cytoxicity, phagocytosis, complement-dependent cytoxicity and half-life/clearance rate. (Presta, Advanced Drug Delivery Reviews 58:640-656, 2006.) Other useful activities include the use of toxic groups that could be delivered to the S. aureus by the binding protein and the use of a second antigen binding protein targeting a host or foreign antigen to enhance stability or activity of a first antigen binding protein targeting the CS-D7 target region.

Antibody effector functions are mediated by different host components, such as Fcγ receptors, neonatal Fc receptor (FcRn), and C1q. (Presta, Advanced Drug Delivery Reviews 58:640-656, 2006, Satoh et al., Expert Opin. Biol. Ther. 6:1161-1173, 2006.) Different types of antibody component or alterations can be used to enhance effector functions. Examples of useful components or alternations include the use of non-fucosylated oligosaccharides, amino acids with enhanced binding to FcRn, and amino acid alterations with enhanced binding to a Fcγ receptor. (Presta, Advanced Drug Delivery Reviews 58:640-656, 2006; Satoh et al., Expert Opin. Biol. Ther. 6:1161-1173, 2006; Lazar et al., U.S. Patent Application Publication US 2004/0132101; Shields et al., The Journal of Biological Chemistry 276:6591-6604, 2001; Dall'Acqua et al., The Journal of Biological Chemistry 281:23514-23524, 2006.)

In one embodiment of the present invention, the antigen binding protein targeting the CS-D7 target region is bispecific. A bispecific antigen binding protein targeting CS-D7 contains two or more binding regions wherein one region targets the CS-D7 target site and a second region targets a different epitope. Additional regions may be present. Examples of general types of bispecific antigen binding proteins include bispecific antibodies and heteropolymers, both of which can be provided in multiple valency such as divalent, trivalent, tetravalent, etc.

In an embodiment, the bispecific antigen binding protein is a bispecific antibody (see, e.g., Marvin and Zhu, Acta Pharmacologica Sinica 26:649-658, 2005; Zuo et al., Protein Engineering 13:361-367, 2000; Ridgway et al., Protein Engineering 9:617-621, 1996; Alt et al., FEBS Letters 454:90-94, 1999; Carter, J. Immunol. Methods 248:7-15, 2001). In a further embodiment, the bispecific antibody targeting the CS-D7 target region also targets a host or foreign antigen. A host antigen can be targeted, for example, to increase stability or activity. Examples of different embodiments related to bispecific antibodies, include but are not limited to, any combination of the following: a bispecific antibody that contains an Fc or modified Fc domain that is capable of mediating antibody effector functions; a bispecific antibody that is bivalent, trivalent or tetravalent; and, a bispecific antibody comprising a second antigen binding protein that specifically binds a C3b-like receptor or another foreign antigen, such a S. aureus or S. epidermidis antigen expressed on the bacterial surface during in vivo infection (e.g., LTA, capsule; O'Riordan and Lee, Clin. Micro. Rev. 17:218-234, 2004; Lees A., KoKai-kun J., LopezAcosta A., Acevedo J., Mond J. 2005. Lipotechoic Acid Conjugate Vaccine for Staphylococcus [abstract]. In: 8^{th} Annual Conference on Vaccine Research; 2005 May 8-11; Baltimore. S1:p. 58; Fischer et al., U.S. Pat. No. 6,610,293; Stinson et al., U.S. Pat. No. 7,250,494).

In another embodiment, the bispecific antigen binding protein targeting the CS-D7 target region can be contained within a heteropolymer complex with a second antigen binding protein targeting a host or foreign antigen. A host antigen can be targeted to enhance stability or activity of the antigen binding protein. Examples of different embodiments include any combination of the following: a heteropolymer containing an Fc or modified Fc domain that is capable of mediating antibody effector functions; a heteropolymer that is bivalent, trivalent or tetravalent; and, heteropolymer comprising a second antigen binding protein that specifically binds a C3b-like receptor or another foreign antigen, such a S. aureus or S. epidermidis antigen expressed on the bacterial surface during in vivo infection. Methods of chemically-crosslinking two antibodies to form a heteropolymeric complex are known in the art. (Taylor et al., Proc, Natl. Acad. Sci. USA 88:3305-3309, 1991; Powers et al., Infection and Immunity 63:1329-1335, 1995.)

Targeting the C3b-like receptors of erythrocytes can help to clear the pathogens from the bloodstream. (see Lindorfer et al., Immunological Reviews 183:10-24, 2001; Mohamed et al., Current Opinion in Molecular Therapeutics 7:144-150, 2005.) The C3b-like receptor in primates is known as either CR1 or CD35 and Factor H in other mammals. Under normal immune adherence conditions, an immune complex (“IC”) comprising a pathogen in association with an antibody that specifically binds the pathogen is tagged with complement proteins (e.g., C3b, C4b) which then bind CR1 on the surface of red blood cells (“RBCs”). The RBCs deliver the IC to phagocytes (e.g., macrophages) expressing Fc receptors (i.e., FcγRs), transferring the IC to the phagocytic cell via interaction between the Fc portion of the IC and the Fc receptor on the cell surface. The IC is then destroyed by the phagocyte, while the RBC returns to circulation. A heteropolymer comprising a bispecific antigen binding protein complex wherein one antigen binding protein is specific for CR1 bypasses the need to activate the complement cascade because the anti-CR1 antibody serves as a surrogate for C3b, the natural ligand for CR1. This can improve the efficiency of the natural immune adherence process for target clearance.

In additional embodiments, bispecific antibodies or heteropolymers target both the CS-D7 target region and CR1, and further contain an Fc constant region. Heteropolymers within this embodiment contain two different antibodies, either or both of the antibodies have an Fc constant region. In one embodiment of the present invention, the anti-CR1 antibody of the heteropolymer specifically binds mouse CR1. In a second embodiment, the anti-CR1 antibody specifically binds human CR1. CR1-specific antibodies are known in the art (see, e.g., Nickells et al., Clin. Exp. Immunol. 112:27-33, 1998).

In another embodiment of the present invention, an antigen binding protein targeting the CS-D7 target region comprises additional components to alter the physiochemical properties of the antigen binding protein, providing significant pharmacological advantages. For example, the attachment of polyethylene glycol (“PEG”) to molecules may help to improve safety and efficiency of said molecules when used as therapeutics. Physiochemical alterations include, but are not limited to, changes in conformation, electrostatic binding, and hydrophobicity which can work together to increase systemic retention of the therapeutic agent. Additionally, by increasing the molecular weight of the antigen binding protein by attaching a PEG moiety, pharmacological advantages include extended circulating life, increased stability, and enhanced protection from host proteases. PEG attachment can also influence binding affinity of the therapeutic moiety to cell receptors. PEG is a non-ionic polymer composed of repeating units (—O—CH₂—CH₂—) to make a range of molecular weight polymers from 400 to greater than 15,000 (e.g., PEG polymers with molecular weights of up to 400,000 are commercially available).

### II.D. EXAMPLES OF DIFFERENT EMBODIMENTS

An antigen binding protein targeting the CS-D7 target region contains a first variable region and a second variable region, wherein the first and second variable regions bind to the target region. Based on the guidance provided herein, antigen binding proteins targeting the CS-D7 target region can be produced having different CDRs and framework amino acids. Additional components such as a hinge region, Fc region, toxic moiety and/or additional antigen binding proteins or binding regions (see Section II.C., supra) may be present.

In a first embodiment concerning the antigen binding protein, the first variable region is a Vₕ region comprising any one, two, or all three of the following CDRs:

a first Vₕ CDR comprising SEQ ID NO: 46 or a sequence differing from SEQ ID NO: 46 by one amino acid; SEQ ID NO: 46 is based on SEQ ID NOs: 35 and 40. SEQ ID NO: 46 has the following amino acid sequence GGSIX¹SSSYYWG, where X¹ is any amino acid. Preferably, X¹ is either serine or arginine;

a second Vₕ CDR comprising either SEQ ID NO: 36, SEQ ID NO: 38, SEQ ID NO: 39, SEQ ID NO: 41, SEQ ID NO: 43 or SEQ ID NO: 44, or a sequence differing from SEQ ID NOs: 36, 38, 39, 41, 43 or 44 by one amino acid; preferably, the second Vₕ CDR comprises either SEQ ID NOs: 36, 38, 39, 41, 43 or 44; and,

a third Vₕ CDR comprising either SEQ ID NO: 37, SEQ ID NO: 42 or SEQ ID NO: 45, or a sequence differing from SEQ ID NOs: 37, 42 or 45 by one amino acid; preferably, the third Vₕ CDR comprises either SEQ ID NOs: 37, 42 or 45.

Preferably, the Vₕ region comprises a first Vₕ CDR(CDR1), a second Vₕ CDR(CDR2) and a third Vₕ CDR(CDR3).

In a second embodiment, the first variable region is a Vₕ region comprising a first, a second and a third CDR which comprise amino acid sequences selected from the group consisting of:

a) SEQ ID NO: 35, SEQ ID NO: 36 and SEQ ID NO: 37, respectively;

b) SEQ ID NO: 35, SEQ ID NO: 38 and SEQ ID NO:37, respectively;

c) SEQ ID NO:35, SEQ ID NO: 39 and SEQ ID NO: 37, respectively;

d) SEQ ID NO: 40, SEQ ID NO: 41 and SEQ ID NO: 42, respectively;

e) SEQ ID NO: 40, SEQ ID NO: 43 and SEQ ID NO: 45, respectively; and,

f) SEQ ID NO: 40, SEQ ID NO: 44 and SEQ ID NO: 42, respectively.

In a further embodiment, the first variable region is a Vₕ region comprising a first Vₕ CDR which comprises SEQ ID NO: 35, a second Vₕ CDR which comprises SEQ ID NO: 36, and a third Vₕ CDR which comprises SEQ ID NO: 37.

In a third embodiment, the second variable region is a Vₗ region comprising any one, two, or all three of the following CDRs:

a first Vₗ CDR comprising either SEQ ID NO: 17, SEQ ID NO: 20, SEQ ID NO: 23, SEQ ID NO: 26, SEQ ID NO: 29 or SEQ ID NO: 32, or a sequence differing from SEQ ID NOs: 17, 20, 23, 26, 29 or 32 by one amino acid; preferably the first Vₗ CDR comprises SEQ ID NOs: 17, 20, 23, 26, 29 or 32;

a second Vₗ CDR comprising either SEQ ID NO: 18, SEQ ID NO: 21, SEQ ID NO: 24, SEQ ID NO: 27, SEQ ID NO: 30 or SEQ ID NO: 33, or a sequence differing from SEQ ID NOs: 18, 21, 24, 27, 30 or 33 by one amino acid; preferably the second Vₗ CDR comprises SEQ ID NOs: 18, 21, 24, 27, 30 or 33; and,

a third Vₗ CDR comprising either SEQ ID NO: 19, SEQ ID NO: 22, SEQ ID NO: 25, SEQ ID NO: 28, SEQ ID NO: 31 or SEQ ID NO: 34, or a sequence differing from SEQ ID NOs: 19, 22, 25, 28, 31 or 34 by one amino acid; preferably the third Vₗ CDR comprises SEQ ID NOs: 19, 22, 25, 28, 31 or 34.

Preferably, the Vₗ region comprises a first Vₗ CDR(CDR1), a second Vₗ CDR(CDR2) and a third Vₗ CDR(CDR3).

In a fourth embodiment, the second variable region is a Vₗ region comprising a first, a second and a third CDR which comprise amino acid sequences selected from the group consisting of:

a) SEQ ID NO: 17, SEQ ID NO: 18 and SEQ ID NO: 19, respectively;

b) SEQ ID NO: 20, SEQ ID NO: 21 and SEQ ID NO: 22, respectively;

c) SEQ ID NO: 23, SEQ ID NO: 24 and SEQ ID NO: 25, respectively;

d) SEQ ID NO: 26, SEQ ID NO: 27 and SEQ ID NO: 28, respectively;

e) SEQ ID NO: 29, SEQ ID NO: 30 and SEQ ID NO: 31, respectively; and,

f) SEQ ID NO: 32, SEQ ID NO: 33 and SEQ ID NO: 34, respectively.

In a further embodiment, the first variable region is a Vₗ region comprising a first Vₕ CDR which comprises SEQ ID NO: 17, a second Vₗ CDR which comprises SEQ ID NO: 18, and a third Vₗ CDR which comprises SEQ ID NO: 19.

In a fifth embodiment, the binding protein contains the Vₕ region as described in the first or second embodiments and the Vₗ region as described in the third or fourth embodiments.

In a sixth embodiment, the antigen binding protein contains a Vₕ region and a Vₗ region, each comprising a first, a second and a third CDR, wherein said first, second and third Vₕ CDRs and said first, second and third Vₗ CDRs comprise amino acid sequences, respectively, selected from the group consisting of:

a) SEQ ID NO: 35, SEQ ID NO: 36, SEQ ID NO: 37, SEQ ID NO: 17, SEQ ID NO: 18 and SEQ ID NO: 19;

b) SEQ ID NO: 35, SEQ ID NO: 36, SEQ ID NO: 37, SEQ ID NO: 20, SEQ ID NO: 21 and SEQ ID NO: 22;

c) SEQ ID NO: 35, SEQ ID NO: 38, SEQ ID NO:37, SEQ ID NO: 23, SEQ ID NO: 24 and SEQ ID NO: 25;

d) SEQ ID NO:35, SEQ ID NO: 39, SEQ ID NO: 37, SEQ ID NO: 26, SEQ ID NO: 27 and SEQ ID NO: 28;

e) SEQ ID NO: 40, SEQ ID NO: 41, SEQ ID NO: 42, SEQ ID NO: 29, SEQ ID NO: 30 and SEQ ID NO: 31;

f) SEQ ID NO: 40, SEQ ID NO: 43, SEQ ID NO: 45, SEQ ID NO: 29, SEQ ID NO: 30 and SEQ ID NO: 31;

g) SEQ ID NO: 40, SEQ ID NO: 44, SEQ ID NO: 42, SEQ ID NO: 29, SEQ ID NO: 30 and SEQ ID NO: 31;

h) SEQ ID NO: 40, SEQ ID NO: 43, SEQ ID NO: 45, SEQ ID NO: 32, SEQ ID NO: 33 and SEQ ID NO: 34; and,

wherein the order of the SEQ ID NOs correspond to Vₕ CDR1, Vₕ CDR2, Vₕ CDR3, Vₗ CDR1, Vₗ CDR2 and Vₗ CDR3. In a further embodiment, Vₕ CDR1, Vₕ CDR2, Vₕ CDR3, Vₗ CDR1, Vₗ CDR2 and Vₗ CDR3 comprise the amino acid sequences SEQ ID NO: 35, SEQ ID NO: 36, SEQ ID NO: 37, SEQ ID NO: 17, SEQ ID NO: 18 and SEQ ID NO: 19, respectively.

In a seventh embodiment, the binding protein is an antibody having one or more variable regions as described in the first through sixth embodiment described above. In a further embodiment, the antibody is an IgG.

In an eighth embodiment, the variable region provided for in embodiments one to seven described above has a framework region with at least a 90% sequence identity to at least one of the mAbs CS-D7, CS-E11, CS-D10, CS-A10, BMV-H11, BMV-E6, BMV-D4, and BMV-C2 light or heavy chain frameworks (see FIGS. 2 and 3).

Sequence identity (also referred to as percent identical) to a reference sequence is determined by aligning a sequence with the reference sequence and determining the number of identical amino acids in the corresponding regions. This number is divided by the total number of amino acids in the reference sequence (e.g., framework region of SEQ ID NO: 1) and then multiplied by 100 and rounded to the nearest whole number. Sequence identity can be determined by a number of art-recognized sequence comparison algorithms or by visual inspection (see generally Ausubel, F M, et al., Current Protocols in Molecular Biology, 4, John Wiley & Sons, Inc., Brooklyn, N.Y., A.1E.1-A.1F.11, 1996-2004). In further embodiments, the sequence identity is at least 95%, or at least 99%, identical to the framework of any one of the mAbs CS-D7, CS-E11, CS-D10, CS-A10, BMV-H11, BMV-E6, BMV-D4, and BMV-C2; or differs from anyone of the mAb framework by 0, 1, 2, 3, 4, 5, 6, 7, 8, 9, 10, 11, 12, 13, 14, 15, 16, 17, 18, 19, or 20 amino acids.

In a ninth embodiment, the antigen binding protein of the present invention is an antibody which comprises a first variable region which is a Vₕ region comprising an amino acid sequence selected from the group consisting of amino acids 1-126 of SEQ ID NO: 2, SEQ ID NO: 4, SEQ ID NO: 6, SEQ ID NO: 8, SEQ ID NO: 10, SEQ ID NO: 12, SEQ ID NO: 14 and SEQ ID NO: 16. In a further embodiment, the Vₕ region comprises amino acids 1-126 of SEQ ID NO: 2.

In a tenth embodiment, the antigen binding protein of the present invention is an antibody which comprises a second variable region which is a Vₗ region comprising an amino acid sequence selected from the group consisting of amino acids 1-108 of SEQ ID NO: 1, SEQ ID NO: 3, SEQ ID NO: 5, SEQ ID NO: 7, SEQ ID NO: 9, SEQ ID NO: 11, SEQ ID NO: 13 and SEQ ID NO: 15. In a further embodiment, the Vₗ region comprises amino acids 1-108 of SEQ ID NO: 1.

In an eleventh embodiment, the antigen binding protein is an antibody containing either:

a) a light chain variable (Vₗ) region comprising amino acids 1-108 of SEQ ID NO: 1 and a heavy chain variable (Vₕ) region comprising amino acids 1-126 of SEQ ID NO: 2;

b) a Vₗ region comprising SEQ ID NO: 3 and a Vₕ region comprising SEQ ID NO: 4;

c) a Vₗ region comprising SEQ ID NO: 5 and a Vₕ region comprising SEQ ID NO: 6;

d) a Vₗ region comprising SEQ ID NO: 7 and a Vₕ region comprising SEQ ID NO: 8;

e) a Vₗ region comprising SEQ ID NO: 9 and a Vₕ region comprising SEQ ID NO: 10;

f) a Vₗ region comprising SEQ ID NO: 11 and a Vₕ region comprising SEQ ID NO: 12;

g) a Vₗ region comprising SEQ ID NO: 13 and a Vₕ region comprising SEQ ID NO: 14; and,

h) a Vₗ region comprising SEQ ID NO: 15 and a Vₕ region comprising SEQ ID NO: 16.

In a further embodiment, the Vₕ region comprises amino acids 1-126 of SEQ ID NO: 2 and the Vₗ region comprises amino acids 1-108 of SEQ ID NO: 1.

In a twelfth embodiment, the binding protein is an antibody described in embodiments seven to eleven above, comprising a heavy chain comprising a hinge, CH₁, CH₂, and CH₃ regions from an IgG₁, IgG₂, IgG₃ or IgG₄ subtype; and a light chain comprising either a human kappa Cₗ or human lambda Cₗ. In a further embodiment, the antibody is a monoclonal antibody.

In a thirteenth embodiment, the binding protein is an antibody wherein the light chain comprises SEQ ID NO: 1 and the heavy chain comprises SEQ ID NO: 2.

In a fourteenth embodiment, the binding protein is an antibody as described in embodiments seven to thirteen above containing one or more of the following: a glycosylation pattern that is either non-fucosylated or substantially (i.e., less than 10% on a molar basis of the carbohydrates that are present) non-fucosylated; one or more amino acid alterations that enhances Fcγ receptor binding; one or more amino acid alterations that enhances neonatal Fc receptor (FcRn) binding; and, one or more amino acid alterations that enhances C1 q binding.

In a fifteenth embodiment, the indicated region (e.g., variable region, CDR region, framework region) described in embodiments one to fourteen above consists, or consists essentially, of an indicated sequence. Reference to “consists essentially” with respect to a region such as a variable region, CDR region, or framework region, indicates the possible presence of one or more additional amino acids at the amino and/or carboxyl termini, where such amino acids do not significantly decrease binding to the target.

In a sixteenth embodiment, the antigen-binding protein described in embodiments one to fifteen above has Vₕ and Vₗ regions providing an affinity K_{D} of 100 nM or less, or a K_{D} of 500 pM or less, to the target antigen. Binding to the target antigen can be determined as described in Example 8.

In a seventeenth embodiment, the antigen binding protein described in embodiments one to sixteen above is joined to at least one or more additional components, including but not limited to a toxic moiety, a molecule(s) to increase physiochemical and/or pharmacological properties of the antigen binding protein, and a second antigen binding protein (see Section II.C., supra). In a further embodiment, the antigen binding protein is a heteropolymer comprising an antigen binding protein targeting the CS-D7 target region chemically-crosslinked to a second antigen binding protein (e.g., an anti-CR1 antibody). In another embodiment, the antigen binding protein has one or more PEG moieties.

Amino acid differences described in the different embodiments, including those providing for differences in sequence identity, can be an amino acid deletion, insertion or substitution. In substituting amino acids to maintain activity, the substituted amino acids should have one or more similar properties such as approximately the same charge, size, polarity and/or hydrophobicity. CDRs, while responsible for binding to a target, can be varied and still retain target specificity. Framework region sequences can also be varied.

The sequence comparison provided in FIGS. 2 and 3 illustrates examples of variations within CDRs and framework regions. Variations in addition to those illustrated in FIGS. 2 and 3 can be produced. In an embodiment concerning amino acid differences, an additional variation is a conserved amino acid substitution. A conservative substitution replaces an amino acid with another amino acid having similar properties. Table 2 provides a list of groups of amino acids, where one member of the group is a conservative substitution for another member.

TABLE 2
Conservative Substitutions

Ala, Val, Ile, Leu, Met
Ser, Thr
Tyr, Trp
Asn, Gln
Asp, Glu
Lys, Arg, His

### II.E. ANTIGEN BINDING PROTEIN COCKTAILS

Antigen binding proteins targeting the CS-D7 target region can be formulated with one or more additional binding proteins targeting a different ORF0657n epitope or a different protein to form an antigen binding protein cocktail. One embodiment of the present invention relates to an antigen binding protein cocktail comprising a combination of at least two antigen binding proteins, or complexes thereof (see, supra, Section II.C.), wherein at least one of the antigen binding proteins targets the CS-D7 target region as described herein. The additional antigen binding proteins are preferably specific to additional S. aureus or S. epidermidis antigens expressed on the bacterial cell surface during in vivo infection, including but not limited to the following: LTA and capsule (O'Riordan and Lee, Clin. Micro. Rev. 17:218-234, 2004; Lees A., KoKai-kun J., LopezAcosta A., Acevedo J., Mond J. 2005. Lipotechoic Acid Conjugate Vaccine for Staphylococcus [abstract]. In: 8^{th} Annual Conference on Vaccine Research; 2005 May 8-11; Baltimore. S1:p. 58; Fischer et al., U.S. Pat. No. 6,610,293; Stinson et al., U.S. Pat. No. 7,250,494); sai-1 related polypeptides (Anderson et al., International Publication No. WO 05/79315); ORF0594 related polypeptides (Anderson et al., International Publication No. WO 05/086663); ORF0826 related polypeptides (Anderson et al., International Publication No. WO 05/115113); PBP4 related polypeptides (Anderson et al., International Publication No. WO 06/033918); AhpC related polypeptides and AhpC-AhpF compositions (Kelly et al. International Publication No. WO 06/078680); S. aureus type 5 and type 8 capsular polysaccharides (Shinefield et al., N Eng. J. Med. 346:491-496, 2002); collagen adhesin, fibrinogen binding proteins and clumping factor (Mamo et al., FEMS Immunology and Medical Microbiology 10:47-54, 1994, Nilsson et al., J. Clin. Invest. 101:2640-2649, 1998, Josefsson et al., The Journal of Infectious Diseases 184:1572-1580, 2001); and, polysaccharide intercellular adhesin and fragments thereof (Joyce et al., Carbohydrate Research 338:903-922, 2003).

In one embodiment, the antigen binding protein contained within the cocktail that targets the CS-D7 target region is a monoclonal antibody as described herein. In another embodiment, each antigen binding protein contained within the antibody cocktail is a monoclonal antibody. In another embodiment, the antigen binding protein cocktail is part of a pharmaceutical composition containing a therapeutically effective amount of said cocktail and a pharmaceutically acceptable carrier.

Thus, included within this portion of the present invention is a cocktail of antigen binding protein complexes (see, supra, Section II.C.) wherein at least one of the antigen binding proteins targets the CS-D7 target region. For example, the present invention further relates to a cocktail of heteropolymer complexes as described in Section II.C. (supra) comprising a combination of at least two heteropolymer complexes, wherein one heteropolymer complex comprises an antigen binding protein that targets the CS-D7 target region, as described herein, chemically-crosslinked to an antibody that specifically binds CR1. This heteropolymer can be combined in the form of an antigen binding protein cocktail with a second heteropolymer complex comprising an antigen binding protein that specifically binds an additional S. aureus antigen expressed on the bacterial cell surface during in vivo infection (e.g., LTA, capsule) chemically-crosslinked to an antibody that specifically binds CR1.

### III. PROTEIN PRODUCTION

Antigen binding proteins and regions thereof are preferably produced using recombinant nucleic acid techniques or through the use of a hybridoma. Different antigen binding proteins can be produced including a single chain protein containing a Vₕ region and Vₗ region such as a single-chain antibody, and antibodies or fragments thereof; and a multi-chain protein containing a Vₕ and Vₗ region a parts of separate proteins.

Recombinant nucleic acid techniques involve constructing a nucleic acid template for protein synthesis. Hybridoma techniques involve using an immortalized cell line to produce the antigen binding protein. Suitable recombinant nucleic acid and hybridoma techniques are well known in the art. (See for example, Ausubel, Current Protocols in Molecular Biology, John Wiley, 2005, Harlow et al., Antibodies, A Laboratory Manual, Cold Spring Harbor Laboratory, 1988.)

### III.A. RECOMBINANT NUCLEIC ACIDS

Recombinant nucleic acids encoding an antigen binding protein can be expressed in a host cell that in effect serves as a factory for the encoded protein. The recombinant nucleic acid can provide a recombinant gene encoding the antigen binding protein that exists autonomously from a host cell genome or as part of the host cell genome.

A recombinant gene contains nucleic acid encoding a protein along with regulatory elements for protein expression. Generally, the regulatory elements that are present in a recombinant gene include a transcriptional promoter, a ribosome binding site, a terminator, and an optionally present operator. A preferred element for processing in eukaryotic cells is a polyadenylation signal. Antibody associated introns may also be present. Examples of expression cassettes and vectors for antibody or antibody fragment production are well known in art. (E.g., Persic et al., Gene 187:9-18, 1997, Boel et al., J. Immunol. Methods 239:153-166, 2000, Liang et al., J. Immunol. Methods 247:119-130, 2001, Tsurushita et al., Methods 36:69-83, 2005.)

Due to the degeneracy of the genetic code, a large number of different encoding nucleic acid sequences can be used to code for a particular protein. The degeneracy of the genetic code arises because almost all amino acids are encoded by different combinations of nucleotide triplets or “codons”. Amino acids are encoded by codons as follows:

A=Ala=Alanine: codons GCA, GCC, GCG, GCU
C=Cys=Cysteine: codons UGC, UGU
D=Asp=Aspartic acid: codons GAC, GAU
E=G1u=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=G1n=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAC

Expression of a recombinant gene in a cell is facilitated using an expression vector. Preferably, the expression vector, in addition to a recombinant gene, also contains an origin of replication for autonomous replication in a host cell, a selectable marker, a limited number of useful restriction enzyme sites, and a potential for high copy number.

If desired, nucleic acid encoding an antibody may be integrated into the host chromosome using techniques well known in the art. (E.g., Ausubel, Current Protocols in Molecular Biology, John Wiley, 2005, Marks et al., International Application Number WO 95/17516, International Publication Date Jun. 29, 1995.)

### III.B. RECOMBINANT NUCLEIC ACID EXPRESSION

A variety of different cell lines can be used for recombinant antigen binding protein expression, including those from prokaryotic organisms (e.g., E. coli, Bacillus sp, and Streptomyces sp. (or streptomycete) and from eukaryotic organisms (e.g., yeast, Baculovirus, and mammalian). (Breitling et al., Recombinant Antibodies, John Wiley & Sons, Inc. and Spektrum Akademischer Verlag, 1999, Kipriyanov et al., Molecular Biotechnology 26:39-60, 2004, Tsurushita et al., Methods 36:69-83, 2005.)

Preferred hosts for recombinant antigen binding protein expression provide for mammalian post translational modifications. Post-translational modifications include chemical modification such as glycosylation and disulfide bond formation. Another type of post-translational modification is signal peptide cleavage.

Glycosylation can be important for some antibody effector functions. (Yoo et al., Journal of Immunological Methods 261:1-20, 2002, Presta, Advanced Drug Delivery Reviews 58:640-656, 2006, Satoh et al., Expert Opin. Biol. Ther. 6:1161-1173, 2006.)

Different types of host cells can be used to provide for efficient post-translational modifications including mammalian host cells and non-mammalian cells. Examples of mammalian host cells include Chinese hamster ovary (CHO), HeLa, C6, PC 12, Human Embryonic Kidney (HEK293) and myeloma cells. Mammalian host cells can be modified, for example, to effect glycosylation. (Yoo et al., Journal of Immunological Methods 261:1-20, 2002, Persic et al., Gene 187:9-18, 1997, Presta, Advanced Drug Delivery Reviews 58:640-656, 2006, Satoh et al., Expert Opin. Biol. Ther. 6:1161-1173, 2006.) Non-mammalian cells can also be modified to provide for a desired glycosylation. (Li et al., Nature Biotechnology 24(2):210-215, 2006.) Glycoengineered Pichia pastoris is an example of such a modified non-mammalian cell. (Li et al., Nature Biotechnology 24(2):210-215, 2006.)

### III.C. EXAMPLES OF DIFFERENT EMBODIMENTS

A nucleic acid comprising one or more recombinant genes encoding for either an antigen binding protein Vₕ region or Vₗ region, or encoding for both of said regions, can be used to produce either a complete binding protein that binds to a CS-D7 target region or a component of the binding protein. A complete binding protein can be provided, for example, by using a single recombinant gene to encode a single chain protein containing a Vₕ region and Vₗ region, such as a single-chain antibody, or by using multiple recombinant genes to, for example, produce the individual Vₕ and Vₗ regions. Additionally, a region of a binding protein can be produced, for example, by producing a polypeptide containing the Vₕ region or the Vₗ region in separate cells.

Thus, the present invention comprises a nucleic acid comprising at least one recombinant gene that encodes an antigen binding protein Vₕ region or Vₗ region, wherein a protein comprising said Vₕ or Vₗ region binds to a CS-D7 target region. In a further embodiment, the nucleic acid comprises two recombinant genes, a first recombinant gene encoding the antibody binding protein Vₕ region and a second recombinant gene encoding the antigen binding protein Vₗ region.

In different embodiments, one or more recombinant genes encode the antigen binding protein, or a Vₕ region or Vₗ region, as described in Section II.D. supra. Preferably, the recombinant gene(s) are expressed in a single-host cell to produce the antigen binding protein. The protein can be purified from the cell.

### IV. APPLICATIONS OF ANTIGEN BINDING PROTEINS

Antigen binding proteins recognizing an appropriate epitope can have therapeutic and other applications. Other applications include using an antigen binding protein recognizing an ORF0657n target region to facilitate the production, characterization, or study of ORF0657n antigens and vaccines. Antigens containing certain ORF0657n regions can be used to provide a protective immune response against S. aureus infection. (Anderson et al., International Publication No. WO 2005/009379, International Publication Date Feb. 3, 2005.)

Techniques for using antigen binding proteins, such as monoclonal antibodies, in the production, characterization, or study of a target protein are well known in the art. (See, for example, Ausubel, Current Protocols in Molecular Biology, John Wiley, 2005, Harlow et al., Antibodies, A Laboratory Manual, Cold Spring Harbor Laboratory, 1988, Harlow et al., Using Antibodies, Cold Spring Harbor Laboratory, Cold Spring Harbor, N.Y., Cold Spring Harbor Laboratory Press, 1999, Lipman et al., ILAR Journal 46:258-268, 2005.)

In an embodiment of the present invention, the presence of an ORF0657n antigen in a solution, bound to a microsphere or on a cell, is determined using an antigen binding protein. The ability of the binding protein to bind to a protein present in the solution or cell can be determined using different techniques such as a Western blot, enzyme-linked immunosorbent assay (ELISA), flow cytometry, and Luminex immunoassay.

### V. TREATMENT

Therapeutic and prophylactic treatment can be performed on a patient using an antigen binding protein binding to an appropriate target region. Therapeutic treatment is performed on those persons infected with S. aureus. Prophylactic treatment can be performed on the general population or a subset of the general population. A preferred subset of the general population is a subset of persons at an increased risk of S. aureus infection.

Therapeutic and prophylactic treatments include methods of protecting or treating a patient against a S. aureus infection comprising the step of administering to the patient an antigen binding protein, as described herein, or a pharmaceutical composition thereof. If desired, the antigen binding protein composition provided herein can be provided as part of a cocktail of antigen binding proteins (see, e.g., Section II.E, supra). In addition, the antigen binding protein compositions can be administered as part of a combination treatment regime wherein additional medicinal substances are also provided. Thus, administration of the antigen binding protein, alone or in combination with additional substances, can take the form of a composition that includes a pharmaceutically active carrier.

Combination therapy can be carried out using antigen binding proteins described herein (see, e.g., Section II, supra) along with one or more additional components having medicinal effects, including but not limited to vaccine antigens or antibiotics. The timing of treatment can be designed to achieve prophylactic and/or therapeutic treatment. For example, the additional component can be administered simultaneously with, or within a short period of time before or after, the antigen binding protein treatment. Administration within a short period of time refers to a time period of within approximately two (2) weeks of when an antigen binding protein is administered, depending on the best treatment regimen for the patient.

Administration of antibiotics effective against S. aureus infection are well known in the art (see, e.g., Anstead et al., Methods Mol. Bio. 391:227-258, 2007; Micek, Clin. Infect. Dis. 45:5184-5190, 2007; Moellering, Clin. Infect. Dis. 46:1032-1037, 2008). Possible antibiotics for combination treatment include, for example: vancomycin, linezolid, clindamycin, doxycycline, rifampin, daptomycin, quinuprintin-dalfopristin, tigecycline, oritavancin, dalbavancin, ceftobiprole, telavancin and iclaprim.

Potential antigens for combination treatment include, for example: ORF0657n related polypeptides (Anderson et al., International Publication No WO 05/009379); sai-1 related polypeptides (Anderson et al., International Publication No. WO 05/79315); ORF0594 related polypeptides (Anderson et al., International Publication No. WO 05/086663); ORF0826 related polypeptides (Anderson et al., International Publication No. WO 05/115113); PBP4 related polypeptides (Anderson et al., International Publication No. WO 06/033918); AhpC related polypeptides and AhpC-AhpF compositions (Kelly et al. International Publication No. WO 06/078680); S. aureus type 5 and type 8 capsular polysaccharides (Shinefield et al., N Eng. J. Med. 346:491-496, 2002); collagen adhesin, fibrinogen binding proteins and clumping factor (Mamo et al., FEMS Immunology and Medical Microbiology 10:47-54, 1994, Nilsson et al., J. Clin. Invest. 101:2640-2649, 1998, Josefsson et al., The Journal of Infectious Diseases 184:1572-1580, 2001); and, polysaccharide intercellular adhesin and fragments thereof (Joyce et al., Carbohydrate Research 338:903-922, 2003)

A “patient” refers to a mammal capable of being infected with S. aureus. Preferably, the patient is a human. However, other types of mammals such as cows, pigs, sheep, goats, rabbits, horses, dogs, cats, monkeys, rats, and mice, can be infected with S. aureus. Treatment of non-human patients is useful in protecting pets and livestock, and in evaluating the efficacy of a particular treatment.

Persons with an increased risk of S. aureus infection include health care workers; hospital patients; patients with a weakened immune system; patients undergoing surgery; patients receiving foreign body implants, such as a catheter or a vascular device; patients facing therapy leading to a weakened immunity; and persons in professions having an increased risk of burn or wound injury. (The Staphylococci in Human Disease, Crossley and Archer (ed.), Churchill Livingstone Inc. 1997.)

In an embodiment, a patient is administered one or more antigen binding proteins in conjunction with surgery or a foreign body implant. Reference to “surgery or a foreign body implant” includes surgery with or without providing a foreign implant, and providing a foreign implant with or without surgery. The timing of administration can be designed to achieve prophylactic treatment and/or therapeutic treatment. Administration is preferably started around the same time as surgery or implantation.

Guidelines for pharmaceutical administration in general are provided in, for example, Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990.

Pharmaceutically acceptable carriers facilitate storage or administration of an antigen binding protein. Substances used to stabilize protein solution formulations include carbohydrates, amino acids, and buffering salts. (Middaugh et al., Handbook of Experimental Pharmacology 137:33-58, 1999.)

Antigen binding proteins can be administered by different routes such as one or more of the following: intraveneous, subcutaneous, intramuscular, and mucosal. Subcutaneous and intramuscular administration can be performed using, for example, needles or jet-injectors. Mucosal delivery, such as nasal delivery, can involve using enhancers or mucoadhesives to produce a longer retention time at adsorption sites. (Middaugh et al., Handbook of Experimental Pharmacology 137:33-58, 1999.)

Suitable dosing regimens are preferably determined taking into account factors well known in the art including age, weight, sex and medical condition of the patient; the route of administration; the desired effect; and the particular antigen binding protein employed. It is expected that an effective dose range should be about 0.1 mg/kg to 20 mg/kg, or 0.5 mg/kg to 5 mg/kg. The dosing frequency can vary depending upon the effectiveness and stability of the compound. Examples of dosing frequencies include biweekly, weekly, monthly and bimonthly.

### VI. CS-D7 TARGET FRAGMENT

A CS-D7 target fragment, present within the ORF0657n region (SEQ ID NO: 47) and bound by mAb CS-D7, can be used, for example, to generate additional antibodies as noted in II.B, supra. A CS-D7 target fragment may also be used to elicit an immune response. Preferably, the CS-D7 target fragment contains the CS-D7 target region. Thus, the CS-D7 target region is provided by ORF0657n. The CS-D7 target fragment appears to be contained within approximately amino acids 42-342 of ORF0657n region (see Example 6) and may be present in smaller fragments derived from this region.

Potential CS-D7 target fragments are provided by different embodiments described below. In a first embodiment, a CS-D7 target fragment is a polypeptide that has at least a 95% sequence identity to portions of ORF0657n (SEQ ID NO:47) selected from the group consisting of amino acids 42-342 of SEQ ID NO: 47, amino acids 42-285 of SEQ ID NO: 47, and amino acids 103-285 of SEQ ID NO: 47, wherein the polypeptide is up to 350 amino acids in length. In additional embodiments concerning the length of the polypeptide, the polypeptide is up to 250 amino acids or up to 200 amino acids. Additional amino acids are preferably additional ORF0657n regions.

In a second embodiment further describing the first embodiment, the SEQ ID NO: 47-related polypeptide is at least 95%, or at least 99%, identical to amino acids 42-342, amino acids 42-285, or amino acids 103-285 of SEQ ID NO: 47; differs from amino acids 42-342, amino acids 42-285, or amino acids 103-285 of SEQ ID NO: 47 by 0, 1, 2, 3, 4, 5, 6, 7, 8, 9, 10, 11, 12, 13, 14, 15, 16, 17, 18, 19, or 20 amino acid alterations; or consists essentially of SEQ ID NO: 47. Each amino acid alteration is independently an amino acid substitution, deletion, or addition. The alterations can be within the SEQ ID NO: 47 region or added to the SEQ ID NO: 47 region.

Reference to “consists essentially” of indicated amino acids indicates that the referred to amino acids are present and additional amino acids may be present. The additional amino acids can be at the carboxyl or amino terminus. In different embodiments 1, 2, 3, 4, 5, 6, 7, 8, 9, 10, 11, 12, 13, 14, 15, 16, 17, 18, 19, or 20 additional amino acids are added to amino acids 42-342, amino acids 42-285, or amino acids 103-285 of SEQ ID NO: 47. An example of an additional amino acid is an amino terminus methionine.

Eliciting an immune response may be useful to help therapeutically or prophylactically treat a S. aureus infection. Immunogens can be formulated and administered to a patient using the guidance provided herein along with techniques well known in the art. Guidelines for pharmaceutical administration in general are provided in, for example, Vaccines Eds. Plotkin and Orenstein, W. B. Sanders Company, 1999; Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990.

Pharmaceutically acceptable carriers facilitate storage and administration of an immunogen to a patient. Pharmaceutically acceptable carriers may contain different components such as a buffer, sterile water for injection, normal saline or phosphate buffered saline, sucrose, histidine, salts and polysorbate.

Immunogens can be administered by different routes such as subcutaneous, intramuscular, or mucosal. Subcutaneous and intramuscular administration can be performed using, for example, needles or jet-injectors.

Suitable dosing regimens are preferably determined taking into account factors well known in the art including age, weight, sex and medical condition of the patient; the route of administration; the desired effect; and the particular compound employed. The immunogen can be used in multi-dose vaccine formats. It is expected that a dose would consist of the range of 1.0 μg to 1.0 mg total polypeptide. In different embodiments the range is from 5.0 μg to 500 μg, 0.01 mg to 1.0 mg or 0.1 mg to 1.0 mg.

The timing of doses depends upon factors well known in the art. After the initial administration one or more booster doses may subsequently be administered to maintain or boost antibody titers. An example of a dosing regime would be day 1, 1 month, a third dose at either 4, 6 or 12 months, and additional booster doses at distant times as needed.

### VII. EXAMPLES

Examples are provided below further illustrating different features of the present invention. The examples also illustrate useful methodology for practicing the invention. These examples do not limit the claimed invention.

### Example 1

### Isolation of Anti-0657n mAbs from scFv Libraries

ScFvs specific to ORF0657n were identified using phage display libraries. The libraries were panned with ORF0657nH. ScFvs were then counter screened to determine whether they bound to ORF0657t. SEQ ID NO: 47 provides an ORF0657n S. aureus sequence. ORF0657t corresponds to amino acids 42-486 of ORF0657n. ORF0657nH corresponds to amino acids 42-609 of ORF0657n. ORF0657nH and ORF0657t were expressed in yeast.

Three Cambridge antibody libraries (BMV: Peripheral blood lymphocytes; CS: Spleen lymphocytes; and DP47: Germline DP47 framework with Vₕ CDR3 and V_{L} of CS library) were screened for scFvs to ORF0657nH (10 μg/ml) using solid phase panning. 190/264 identified were ORF0657t specific. 172/264 were identified after removing duplicates from variable region DNA sequencing. DNA sequence analysis identified 41 unique sequences from the DP47 screen, 57 sequences from the BMV screen and 74 sequences from the CS library screen after removal of duplicate sequences and sequences containing stop codons.

Phage ELISA Screening—To validate the antigen specificity of the selected scFv-phage clones, 172 phage clones from each library in round 2 and 88 clones from each third round library were tested in an ELISA. A set of the ELISA positives were further tested by flow cytometry and in a competitive luminex assay. ScFvs that bound to ORF0657n on the cell surface of S. aureus as measured by flow cytometry were converted to full IgGs, as described below.

Flow Cytometry Screening—Flow cytometry analysis was preformed to determine the ORF0675n binding site of different ScFvs. S. aureus used for the analysis was grown in an iron-deficient defined medium (RPMI 1640). Over 100 different scFvs isolated from round 2 of solid phase phage display panning were examined by flow cytometry against 2×RPMI grown S. aureus Becker cells. Most of the scFvs tested displayed varying degrees of binding. Data generated from these experiments confirmed that scFvs isolated from panning bound S. aureus.

IgG Conversion—Twelve scFvs clones were identified for IgG conversion. The sequences for the variable regions were PCR amplified and DNA encoding the heavy chain variable regions were fused in-frame with DNA encoding the IgG1 constant region, whereas DNA encoding the light chain variable region were fused in-frame with DNA encoding the corresponding constant region. The cloning procedure for the resulting antibody expression vectors is described below.

The expression of both light and heavy chains was driven by human CMV promoter and bovine growth hormone polyadenylation signal. The leader sequence in the front mediated the secretion of antibodies into the culture medium. The heavy chain leader sequence was MEWSWVFLFFLSVTTGVHS (SEQ ID NO: 49). The light chain leader sequence was MSVPTQVLGLLLLWLTDARC (SEQ ID NO: 50). The expression vectors carry oriP from EBV viral genome for prolonged expression in 293EBNA cells and the bacterial sequences for kanamycin selection marker and replication origin in E. coli.

The variable regions were PCR amplified. PCR reactions were carried out in a volume of 25 μl containing high fidelity PCR master mix, a template volume of 1 and forward and reverse primers: 1 μl each. PCR condition was 1 cycle of 94° C., 2 minutes; 25 cycles of 94° C., 1.5 minutes; 60° C., 1.5 minutes; 72° C., 1.5 minutes and 72° C., 7 minutes; 4° C. until removed and cloned in-frame with leader sequence at the 5′-end and constant region at the 3′-end using In-Fusion strategy. For example, the clone CS-D7 antibody was cloned using the following primers: (light chain forward, 5′-ACAGATGCCAGATGCGAAATTGTGATGACACAGTCT (SEQ ID NO: 51); light chain reverse, 5′-TGCAGCCACCGTACGTTTAATCTCCAGTCGTGTCCC (SEQ ID NO: 52); heavy chain forward, 5′-ACAGGTGTCCACTCGCAGGTGCAGCTGCAGGAGTCG (SEQ ID NO: 53) and heavy chain reverse, 5′-GCCCTTGGTGGATGCACTCGAGACGGTGACCAGGGT (SEQ ID NO: 54)).

The rest of the clones were converted in a similar fashion. The DNA sequences for all the clones were confirmed by sequencing. As further described below, mAbs BMV-H11, BMV-D4, BMV-E6, BMV-C2, CS-D7, CS-D10, CS-A10 and CS-E11 compete for binding to the same epitope using the Luminex binding assay.

The full amino acid sequences for antibody CS-D7 deduced from DNA sequences are shown below in Table 3. The variable regions are shown in bold with the CDRs being underlined.

A sequence comparison for the mAbs BMV-H11, BMV-D4, CS-D7, CS-D10, CS-A10, CS-E11, BMV-E6 and BMV-C2 light and heavy chain variable regions is provided in FIGS. 2 and 3. The sequences for the heavy chain constant regions are all identical, and the sequences for the light chain constant regions are either kappa or lambda. MAb CS-D7 contains the kappa sequence, which corresponds to amino acids 109-201 of SEQ ID NO: 1. MAbs BMV-H11, BMV-D4, CS-D10, CS-A10, CS-E11, BMV-E6 and BMV-C2 contain the lambda sequence which is provided by SEQ ID NO: 48.

The antibodies were expressed in 293EBNA monolayer cells. The plasmids were transfected using PEI based transfection reagents. The transfected cells were incubated in Opti-MEM serum free medium and the secreted antibodies were purified from medium using protein A/G affinity chromatography. The concentration of purified antibodies was determined by OD280 nm and the purity by LabChip capillary electrophoresis.

TABLE 3
mAb CS-D7 Amino Acid Sequences

```
mAb CS-D7 Light Chain Amino Acid Sequence (SEQ ID NO. 1)
    1 EIVMTQSPAT LSVSPGERAT LSCRASQYVS DNLAWYQQKP GQAPRLLIYG
  51 ASTRATGVPA RFSGSGSGTE FTLTISSLQS EDFAVYYCQQ YNNWRPVTFG
101 QGTRLEIKRT VAAPSVFIFP PSDEQLKSGT ASVVCLLNNF YPREAKVQWK
151 VDNALQSGNS QESVTEQDSK DSTYSLSSTL TLSKADYEKH KVYACEVTHQ
201 GLSSPVTKSF NRGEC
mAb CS-D7 Heavy Chain Amino Acid Sequence (SEQ ID NO: 2)
    1 QVQLQESGPG LVKPSETLSL TCTVSGGSIR SSSYYWGWFR QTPGKGLEWL
  51 GNVFFSGSAY YNPSLKNRVT ISIDTSENQS SLKLTSVTAA DTAVYYCARP
101 QAYSHDSSGH SPFDLWGRGT LVTVSSASTK GPSVFPLAPS SKSTSGGTAA
151 LGCLVKDYFP EPVTVSWNSG ALTSGVHTFP AVLQSSGLYS LSSVVTVPSS
201 SLGTQTYICN VNHKPSNTKV DKRVEPKSCD KTHTCPPCPA PELLGGPSVF
251 LFPPKPKDTL MISRTPEVTC VVVDVSHEDP EVKFNWYVDG VEVHNAKTKP
301 REEQYNSTYR VVSVLTVLHQ DWLNGKEYKC KVSNKALPAP IEKTISKAKG
351 QPREPQVYTL PPSREEMTKN QVSLTCLVKG FYPSDIAVEW ESNGQPENNY
401 KTTPPVLDSD GSFFLYSKLT VDKSRWQQGN VFSCSVMHEA LHNHYTQKSL
451 SLSPGK
```

### Example 2

### Luminex Binding Studies

A selection of the CAT scFvs (see Example 1) were characterized by screening against a panel of murine mAbs (2H2, 13C7, 1G3, and 13G11) and CAT mAbs (CS-D3, CS-D7, CS-D10, CS-E11, BMV-E6, BMV-D4, mAb 1 and mAb 2) to determine whether they competed for the same epitopes on ORF0657n or bound to different epitopes. In these assays a single CAT scFv (or CAT mAb) was competed against a single mAb (Murine or CAT).

Murine antibodies 2H2, 1G3, 13C7 or 13G11 are described in International application PCT/U.S.07/01687, filed Jan. 23, 2007, hereby incorporated by reference herein. PCT/U.S.07/01687 (International Publication Number WO 2007/089470) refers to hybridoma cell lines producing mAb 1G3.BD4, mAb 2H2.BE11, mAb 13C7.BC1, and mAb 13G11.BF3 being deposited with the American Type Culture Collection, 10801 University Boulevard, Manassas, Va. 20110-2209, in accordance with the Budapest Treaty on Sep. 30, 2005. The cells lines were designated: ATCC No. PTA-7124 (producing mAb 2H2.BE11), ATCC No. PTA-7125 (producing mAb 13C7.BC1), ATCC No. PTA-7126 (producing mAb 1G3.BD4), and ATCC No. PTA-7127 (producing mAb 13G11.BF3).

Construction of ORF0657n-bead—9.4×10⁶ Radix maleimide microspheres (Georgetown, Tex.) were coupled to 750 μg ORF0657n-Se (ORF0657n containing carboxyl terminal cysteine group) at room temperature for 2 hours. Beads were washed 3 times with 1 ml PBS and then quenched with 1 M N-acetyl-L-cysteine (Sigma) for 2 hours at room temperature. Microspheres were washed 3× in PBS. Beads were enumerated and re-suspended at a final concentration of 500 microspheres/μl.

Detection of Competition between CAT ScFvs and Murine mAbs—CAT scFvs were diluted 1:4, 1:8, 1:16 and 1:32 in PBS-TBN (0.05% Tween 20, 1% BSA and 0.05% sodium azide) and incubated with 5000 ORF0657n-coupled microspheres in a Multiscreen filter plate (Millipore) for 1 hour, 15 minutes at room temperature. The beads were then washed 3× with PBS with 0.05% Tween 20 (PBS/Tween20). ORF0657n-beads with bound CAT scFv were incubated with murine monoclonal antibodies (2H2, 1G3, 13C7 or 13G11). The murine mAbs had been commercially labeled with a R-phycoerythrin (PE) conjugate (Chromoprobe Inc.). The labeled mAbs were separately diluted in PBS-TBN to a final concentration of 2 μg/ml. The diluted mAbs (50 μl, per well) were added to plates which were incubated an additional 1 hour, 15 minutes at room temperature. Microspheres were washed 3× with PBS/Tween20. Microspheres were re-suspended in PBS/Tween20 and the median fluorescent signal was read using a Bio-Plex luminometer (BioRad).

Based on binding competition between scFvs with the murine mAbs, the binding site of the tested antibodies was divided into the following groups. Group 1, the scFvs BMV-C2, BMV-E6, BMV-D4, BMV-H11, CS-E11, CS-A10, CS-D10, and CS-D7, did not compete with any of the murine mAbs; Group 2, two scFvs competed with the murine mAb 2H2; Group 3, two scFvs competed with mAb 1G3; Group 4 none of the scFvs competed with mAb 13C7; and Group 5, none of the scFvs competed with mAb 13G11. For scFvs CS-D10 and CS-D7, the results using Biacore were different from the analysis in the present study using Luminex (see Example 5, infra).

Detection of Competition between CAT mAbs and Murine mAbs—ScFvs BMV-D4, CS-D7, CS-D10, CS-A10, and BMV-C2, the scFvs competing with murine mAb 2H2, and the two scFvs competing with 1 G3 were converted to IgG antibodies as described in Example 1. CAT mAbs were diluted to a concentration of 2 μg/ml in PBS-TBN and incubated with 5000 ORF0657n-coupled microspheres in a Multiscreen filter plate (Millipore) for 1 hour, 15 minutes at room temperature. The beads were then washed 3× with PBS with 0.05% Tween 20 (PBS/Tween20). ORF0657n-beads with bound CAT mAbs were incubated with murine monoclonal antibodies (2H2, 1G3, 13C7 or 13G11). The murine mAbs had been commercially labeled with a R-phycoerythrin (PE) conjugate (Chromoprobe Inc.). The labeled mAbs were separately diluted in PBS-TBN to a final concentration of 2 μg/ml. The diluted mAbs (50 μL per well) were added to plates which were incubated an additional 1 hour, 15 minutes at room temperature. Microspheres were washed 3× with PBS/Tween20. Microspheres were re-suspended in PBS/Tween20 and the median fluorescent signal was read using a Bio-Plex luminometer (BioRad).

The CAT mAbs were tested for competition against the murine mAbs. The same five groups were observed as with the scFvs and murine mAbs competition described above.

Detection of Competition between CAT ScFvs and CAT mAbs—CAT scFvs were individually competed against the CAT antibodies in the Luminex cLIA assay. ScFvs were diluted 1:4, 1:8, 1:16 and 1:32 in PBS-TBN (0.05% Tween 20, 1% BSA and 0.05% sodium azide) and incubated with 5000 ORF0657n-coupled microspheres in a Multiscreen filter plate (Millipore) for 1 hour, 15 minutes at room temperature. The beads were then washed 3× with PBS with 0.05% Tween 20 (PBS/Tween20). The individual antibodies were diluted in PBS-TBN to a final concentration of 2 μg/ml and added to separate plates. 50 μL/well of the dilute antibody were added to the plates which were incubated an additional 1 hour, 15 minutes at room temperature. The plates were washed 3× with PBS/Tween20. Biotrend's anti-human IgG (Fc specific) antibody HP6043 (R-phycoerythrin labeled) was diluted 1:50. 50 μL/well of dilute antibody was added to the plate. The plates were incubated at room temperature for 1 hour and 15 minutes. The microspheres were washed 3× with PBS-Tween20. Microspheres were re-suspended in PBS/Tween20 and the median fluorescent signal was read using a Bio-Plex luminometer (BioRad). ScFvs were considered competitive if the median fluorescent signal was reduced by at least 30% over the signal detected for non-competitive scFvs for at least two dilutions.

The outcome of the competition for the individual scFvs BMV-H11, BMV-D4, BMV-E6, BMV-C2, CS-D7, CS-D10, CS-A10, and CS-E11 versus the individual mAbs mAb BMV-D4, BMV-E6, CS-D7, CS-D10, and CS-E11 are shown in Table 4. Each scFv in the first column was found to compete individually against all the mAbs provided in the second column.

TABLE 4
CAT scFV competing mAb

| scFV | mAb |
|---|---|
| CS-D7, BMV-H11, BMV-D4, CS- | CS-D7, BMV-D4, CS-D10, |
| D10, CS-A10, CS-E11, BMV-E6, | CS-E11, BMV-E6 |
| BMV-C2 | |

### Example 3

### BIACORE® Measurement of Binding to ORF0657n

Antibody binding to ORF0657n was determined by BIACORE®. BIACORE® incorporates microfluidics technology and surface plasmon resonance (SPR) to detect changes in mass by monitoring changes in the refractive index of a polarized light aimed directly at the surface of a carboxylmethyl dextran coated (CM5) sensor chip. The changes in response, measured in Response Units, can be correlated to the amount of bound analyte (e.g., antigen or antibody).

Affinity binding to ORF0657n was measured by BIACORE® using the anti-Staph antibody mAb 13C7.D12. The antibody was covalently bound on the surface of the CM5 sensor chip. The bound Ab was exposed first to the ORF0657n and subsequently to antibodies being tested at low concentration (5 μg/mL). After each cycle of ORF0657n+antibody, the surface of the sensor chip was regenerated back to the immobilized 13C7.D12 using 20 mM HCl.

To normalize for the amount of antigen initially bound (captured) in each run, the following ratio for each test antibody/antigen complex is calculated:

$$= \frac{Test Antibody Response Units * 1000}{0657 n protein Response Units} or \frac{{mRu}_{Ab}}{{RU}_{Ag}}$$

The results are shown in Table 5. No significant binding was exhibited for mAb CS-D4 and mAb CS-D6. Reasons for lack of IgG binding include absence or incorrect value for IgG protein, antibody aggregation, very poor binding activity of the IgG or complete overlap with the capture antibody (when the antigen is only present in monomeric form). mAb CS-D4 and mAb CS-E6 binding were observed in a pairwise binding study, where the antibody concentration was increased significantly (Example 4, infra.)

TABLE 5

| | mRU Ab/RU Ag | | | |
|---|---|---|---|---|
| mAb | Replicate 1 | Replicate 2 | Average | % Difference |
| CS-D7 | 295 | 303 | 299 | 3 |
| CS-D10 | 83 | 86 | 189 | 2 |
| 1G3.BD4 | 78 | 82 | 80 | 4 |
| 2H2-BE11 | 51 | 37 | 44 | 32 |
| BMV-D4 | 0 | 0 | — | — |
| BMV-E6 | 0 | 0 | — | — |

### Example 4

### Pair-Wise Competition Binding

Pair-wise binding experiments were conducted to determine the relative binding of antibodies to the ORF0657n. The anti-Staph antibody mAb 13C7.D12 was covalently bound (immobilized) on the surface of the CM5 sensor chip. The immobilized Ab was exposed first to the ORF0657n and subsequently to a pair of antibodies in a matrix format. After each cycle of 0657n protein+antibody pair, the surface of a sensor chip was regenerated back to the immobilized 13C7.D12 using 20 mM HCl. Antibodies were tested against the ORF0657n protein in a matrix format so that all combinations of each antibody pair could be analyzed.

The matrix design for mAbs CS-D7, CD-D10, BMV-D4, and BMV-E6 is summarized in Table 6.

TABLE 6

| | Second Antibody | | | |
|---|---|---|---|---|
| First Antibody | Flow Cell 1 | Flow Cell 2 | Flow Cell 3 | Flow Cell 4 |
| CS-D7 | CS-D7 | CS-D10 | BMV-D4 | BMV-E6 |
| CS-D10 | CS-D7 | CS-D10 | BMV-D4 | BMV-E6 |
| BMV-D4 | CS-D7 | CS-D10 | BMV-D4 | BMV-E6 |
| BMV-E6 | CS-D7 | CS-D10 | BMV-D4 | BMV-E6 |

To normalize for the amount of antigen initially bound (captured) in each run, the following ratio for each test antibody/antigen complex is calculated:

$$= \frac{Test Antibody Response Units * 1000}{0657 n protein Response Units} or \frac{{mRu}_{Ab}}{{RU}_{Ag}}$$

The percentage of available epitope remaining for each antibody can be calculated for the mapping pair as follows:

$$= \frac{( {mRU}_{Ab} ( when {2}^{nd} Ab ) / {RU}_{Ag} ) * 100}{( {mRU}_{Ab} ( when {1}^{st} Ab ) / {RU}_{Ag} ) } or \begin{matrix}\% Remaining \\ ( calculated for each Ab )\end{matrix}$$

The monoclonal antibodies CS-D7, CS-D10, BMV-D4 and BMV-E6 were directed to the same or significantly overlapping regions. Table 7 summarizes results of the pair wise binding study.

TABLE 7

| Second antibody | First antibody to bind | | | |
|---|---|---|---|---|
| to bind | CS-D7 | CS-D10 | BMV-D4 | BMV-E6 |
| CS-D7 | 0-25% | 26-50% | 26-50% | 26-50% |
| CS-D10 | 0-25% | 0-25% | 26-50% | 26-50% |
| BMV-D4 | 0-25% | 0-25% | 0-25% | 26-50% |
| BMV-E6 | 0-25% | 0-25% | 0-25% | 0-25% |

The indicated percent is the percent of epitope available for 2^{nd} antibody binding.

Relative footprint size of the mAbs is as follows: CS-D7>CS-D10>BMV-D4>BMV-E6. The monoclonal antibody CS-D7 had the largest “footprint” (highest ratio mRU Ab/RUAb when it is the first antibody to bind). In contrast to the binding studies described in Example 3, antibody concentration was increased significantly and binding was observed using mAb BMV-D4 and mAb BMV-D6.

### Example 5

### Additional Epitope Foot Printing

Additional epitope foot printing studies were done using BIACore for a subset of the CAT ScFvs and mAbs. In these studies the Group One and Group Two binding competition was the same as in Example 2. However, CS-D7 and CS-E7 were found to compete with mAb 1G3 (Group 3).

Antibodies were assayed for epitope overlap on a BIACore 2000, and all reagents were supplied by BIACore (Piscataway, N.J.) unless otherwise specified. For each experiment, flow cells were activated with an EDC/NHS (NHS, N-hydroxysuccinimide; EDC, (N-ethyl-N′-(3-dimethylaminopropyl)carbodiimide) mixture; various monoclonal antibodies were injected over these activated surfaces in sodium acetate pH 5.0, and then the surfaces were blocked with 1.0 M ethanolamine-HCl pH 8.5. In each experimental cycle, 150 μL of ORF0657t was injected over the covalently immobilized monoclonal antibodies at 10 μL/min. Subsequently, 120 μL of another monoclonal antibody was injected over the conjugates at 10 μL/min. Throughout the experiment, surface plasmon resonance data were acquired and stored. Regenerations were performed with a single injection of 20 mM HCl flowing at 60 μL/min with a contact time of 30 seconds. All experimental steps were performed under a controlled temperature of 25° C. Subsequent analyses were performed with internally-developed software running on the Matlab platform (7.2.0.232 R2006a, Mathworks Inc., Natick, Mass.).

As noted in Example 2, in competitive Luminex assays mAb CS-D7 does not compete with mAb 1G3. The competitive Luminex and sequential BIACore assays have differences that permit this discrepancy. First, the competitive bead-based Luminex assay allows one antibody to displace another, while the surface plasmon resonance (SPR) assay on the Biacore 2000 has one antibody covalently (irreversibly) coupled. Second, the competitive assay uses a label specific to one of the antibodies, while the SPR assay is label-free and sensitive only the amount of material on the surface. In the competitive assay, a labeled high affinity antibody could displace a pre-bound low affinity antibody, causing a false result of no epitope overlap. In either assay, where both antibodies bind without interference, then no overlap exists. However, in the converse case, steric hindrances could cause an apparent epitope overlap when the epitopes may be different. This effect may be enhanced in both assays where the binding steps are sequential and not simultaneous.

A distinction between the experiments in Examples 3 and 4, and Example 5, is that in Examples 3 and 4, ORF0657n is first captured with 13C7 mAb and then another pair of antibodies is sequentially flowed to see if they can bind to ORF0657n. If either of these antibodies shares an epitope with mAb 13C7 then it may not bind to ORF0657n. Thus, it is effectively a 3-way comparison between mAb 13C7, “first antibody”, and “second antibody”. In contrast, Example 5 is a 2-way comparison.

### Example 6

### mAb CS-D7 Epitope Mapping

Epitope mapping of the mAb CS-D7 target region was performed by chemical cleavage of the linear sequence of ORF0657t and determining which fragments were bound by mAb CS-D7. ORF0657t was chemically cleaved with CNBr for 2 hours. The resulting cleavage products were analyzed by SDS PAGE. SDS PAGE analysis showed 10 bands with molecular weights of approximately 47, 44, 37, 35, 32, 26, 16, 13 and 10 kDa. A Western Blot analysis with mAb CS-D7 clearly showed that only the 47, 44, 37, 35 and 32 kDa bands were recognized by mAb CS-D7. The absence of short sequences that are recognized by mAb CS-D7 indicates that mAb CS-D7 does not recognize a linear sequence of ORF0657n.

The Western Blot bands were excised from the SDS PAGE gel, in-gel digest was performed, and the resulting peptides that were identified by tandem mass spectrometry matched to corresponding sequences in ORFO657n. The results are shown in Table 8.

TABLE 8

| SDS | | | | | |
|---|---|---|---|---|---|
| PAGE | Apparent MW | Western | Western | ORF0657n | Calculated |
| band | (SDS PAGE) | positive | negative | [amino acids] | MW |
| 1 | 47 kDa | X | | [42-396] | 40.7 kDa |
| 2 | 44 kDa | X | | [42-362] | 36.5 kDa |
| 4 | 37 kDa | X | | [42-342] | 34.1 kDa |
| 5 | 35 kDa | X | | [42-342] | 34.1 kDa |
| | | | | [156-486] | 38.5 kDa |
| 6 | 32 kDa | X | | [42-342] | 34.1 kDa |
| 7 | 26 kDa | | X | [42-254] | 23.8 kDa |
| | | | | [255-486] | 26.9 kDa |
| 8 | 16 kDa | | X | [254-384] | 15.3 kDa |
| | | | | [254-396] | 16.8 kDa |
| | | | | [343-486] | 16.6 kDa |
| 9 | 13 kDa | | X | [156-254] | 11.5 kDa |
| | | | | [255-375] | 14.2 kDa |
| | | | | [375-486] | 12.9 kDa |
| 10 | 10 kDa | | X | [156-254] | 11.5 kDa |
| | | | | [255-342] | 10.3 kDa |
| | | | | [397-486] | 10.1 kDa |

The indicated ORF0657n regions in Table 8 are based on the following: peptides identified in the in-gel digest, C-terminal methionine residues identified by tandem mass spectra, the assumption that all fragments of the CNBr digest start and end with a methionine cleavage site, and the apparent molecular weight of the band in the SDS PAGE gel. The smallest internal fragment of ORF0657t that could be identified by mAb CS-D7 in a Western Blot analysis was amino acids 42-342.

### Example 7

### mAb CS-D7 Epitope Excision

The requirement of a higher molecular weight fragment of ORF0657t for binding to mAb CS-D7 was confirmed by epitope excision. In detail, mAb CS-D7 was immobilized by chemical cross linking to cyanogen bromide activated sepharose (Amersham cat no 17 0430 01) for each of the epitope excision experiments. Then intact ORF0657t was allowed to bind to the immobilized antibody and non-bound ORF0657t washed off by intensive washing with phosphate buffered saline. Trypsin was added to the bound ORF0657t. Peptides that were excised by the proteases during the incubation were thoroughly washed away and ORF0657t fragments that specifically bound to mAb CS-D7 were released with SDS loading buffer.

Fragments specifically bound to mAb CS-D7 were analyzed by SDS PAGE. The epitope excision experiment showed three bands with molecular weights of approximately 48, 23 and 19.5 kDa in the SDS PAGE analysis. All bands were excised from the SDS PAGE gel, in-gel digests were performed, and peptides of ORF0657n that correspond to the bands were identified by tandem mass spectrometry (Table 9). The calculated molecular weight of each ORF0657n peptide identified by tandem mass spectrometry is smaller than the molecular weights of the corresponding bands identified by SDS-PAGE (Table 9), a consequence of the experimental design. Thus, it is likely that the fragments bound to mAb CS-D7 in this experiment actually span larger polypeptide regions of ORF0657n than those identified by mass spectrometry. For example, Band 3 is identified by mass spectrometry as corresponding to amino acids 117-224 of ORF0657n and has a calculated molecular weight of 12.5 kDa. It has a molecular weight as identified by SDS-PAGE of 19.5 kDa. If an amino acid region corresponding to approximately 7 kDa (the difference between the calculated and SDS PAGE-identified molecular weights) is added to both the N- and C-terminal portions of the mass spectrometry-identified peptide, a fragment corresponding to approximately amino acids 42-285 of ORF0657n will result. In this case, since a 7 kDa region is added to both the N- and C-terminal portions of the identified fragment, it is likely that the minimal portion of ORF0657n that is necessary to achieve mAb CS-D7 binding corresponds to a region within amino acids 42-285. Since the chemical cleavage experiments in Example 6 indicate that a fragment corresponding to amino acids 42-254 does not bind mAb CS-D7, it is likely that a region corresponding to amino acids 254-285, or a portion thereof, is important for proper antibody binding.

TABLE 9

| | ORF0657n region | | |
|---|---|---|---|
| | identified by tandem | Calculated | |
| | mass spectrometry | molecular | SDS PAGE |
| Band | [amino acids] | weight | band size |
| 1 | [117-456] | 39.7 kDa | 47.7 kDa |
| 2 | [117-196] | 9.2 kDa | 23.3 kDa |
| 3 | [117-224] | 12.5 kDa | 19.5 kD |

### Example 8

### Affinity Determination

Surface plasmon resonance (SPR) evaluation of the scFv CS-D7 and full length IgG CS-D7 were performed using Biacore. To measure a 1:1 interaction between the binding domain and the antigen, experimental set up on Biacore was modified depending on whether antibody fragment or full length IgG was analyzed. For IgG measurements, the IgG was captured to the surface as ligand and ORF0657t was run as analyte. For antibody fragment analysis 0657t was bound to the surface as ligand and the antibody fragment was run as the analyte. Comparison of the two methods yielded similar results (Table 10). Standard deviation was derived from 2 independent experiments for each.

TABLE 10

| Antibody | K_{D} |
|---|---|
| D7 scFv | 179 pM ± 5 pM |
| D7 IgG | 422 pM ± 11 pM |

### Example 9

### Indwelling Catheter Model

Monoclonal antibodies were tested for efficacy in an indwelling catheter model run in rats. Sprague Dawley rats were purchased with indwelling catheters (PESO silicone rubber) surgically implanted into the jugular vein, held in place with sutures, and exiting, with a port, on the dorsal midline of the rat. The rats were housed for >7 days prior to the beginning of an experiment. To test antibody protection of indwelling catheters from colonization by S. aureus, rats were injected ip with 0 to 4 mg of mAb one hour prior to challenge. Animals were challenged with 4×10⁹ CFU through the tail vein. Twenty-four hours later, animals were sacrificed and catheters removed using sterile technique. Catheters (the whole catheter with the external port removed) were placed on mannitol salt agar or TSA (Teknova) for evaluation of colonization. Plates were cultured for 24-48 hours at 37° C. Any sign of colony outgrowth was scored as culture positive. The results of 5 separate experiments are shown in Tables 11 and 12. The p value comparing the 4 mg dose of mAb CS-D7 to the 4 mg dose of mAb 20C2HA (isotype control) in Table 11 is <0.0001. The p value comparing the 4 mg dose of mAb CS-D7 to the PBS control in Table 11 is <0.0001.

TABLE 11
Rat Indwelling Catheter Model (Exps 1-4)

| | Number of Culture - Negative Catheters | | | | |
|---|---|---|---|---|---|
| mAb (quantity) | Exp#1 | Exp#2 | Exp#3 | Exp#4 | Total |
| CS-D7 (0.2 mg) | 0 of 3 (0%) | nd | nd | nd | 0 of 3 (0%) |
| CS-D7 (2 mg) | 0 of 3 (0%) | 1 of 3 (33%) | 2 of 3 (33%) | nd | 3 of 9 (33%) |
| CS-D7 (4 mg) | 3 of 3 (100%) | 3 of 3 (100%) | 3 of 3 (100%) | 3 of 3 (100%) | 12 of 12 (100%) |
| 20C2HA (0.2 mg) | 1 of 3 (33%) | nd | nd | nd | 1 of 3 (33%) |
| (isotype control) | | | | | |
| 20C2HA (2 mg) | 0 of 3 (0%) | 1 of 3 (33%) | 0 of 3 (0%) | nd | 1 of 9 (11%) |
| 20C2HA (4 mg) | 0 of 3 (0%) | 0 of 3 (0%) | 0 of 3 (0%) | 0 of 3 (0%) | 0 of 12 (0%) |
| PBS | 0 of 2 (0%) | 0 of 2 (0%) | 0 of 2 (0%) | 0 of 2 (0%) | 0 of 8 (0%) |

TABLE 12
Rat Indwelling Catheter Model (Exp. 5)

| | Number of Culture - | |
|---|---|---|
| mAb (4 mb) | Negative Catheters | p value |
| CS-D7 | 4 of 5 (80%) | 0.0036 (compared to 20C2HA) |
| 20C2HA | 0 of 5 (0%) | |
| (isotype control) | | |
| PBS | 1 of 5 (20%) | 0.0496 (compared to CS-D7 |

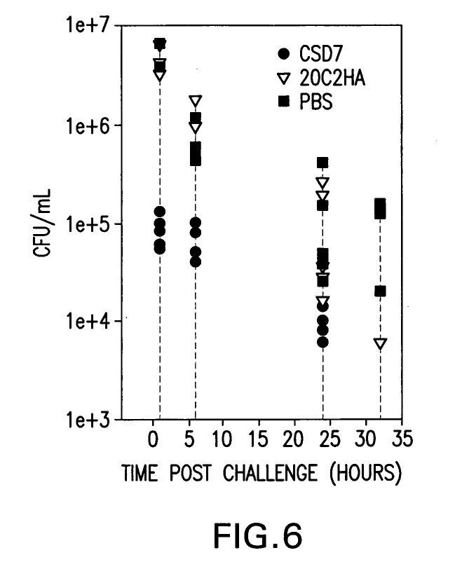

The cannulated rats studied in Experiment 5 (Table 12) were injected ip with 4 mg monoclonal antibody (CS-D7 or isotype control), or PBS alone, 1 hour prior to challenge. They were then challenged iv with 1−2×10⁹ CFU S. aureus strain Becker. Blood was drawn from all rats at the designated time points. At the final time point (32 hours), blood was drawn, and the animals were sacrificed and the catheter removed. Blood was evaluated for bacteria by spreading 50 uL on TSA plates and culturing overnight. The catheters were evaluated for S. aureus by plating on mannitol salt plates overnight. As shown in FIG. 6, a reduction of blood CFU was demonstrated with injection of mAb CS-D7.

### Example 10

### Ex Vivo Model

Monoclonal antibodies were evaluated using a method of passive protection. Bacteria were pre-opsonized ex vivo with mAb prior to lethal injection via the intra-peritoneal (ip) route (ex vivo method). A quantity of bacteria sufficient for 6 Balb/c mice (6×LD₁₀₀) was incubated with 800 μg IgG at 4° C. for 1 hour, with gentle rocking. Bacteria were then pelleted and any unbound mAb removed. Antibody-opsonized bacteria were re-suspended in 2.4 mL of PBS, and 0.4 mL (1×LD₁₀₀) was injected into each of five mice. After challenge, each inoculum was quantitated by plating on TSA to insure that equivalent CFU was given to all groups of mice and that the mAbs had not aggregated the bacteria. Survival was monitored for 3 days post challenge. Since the target antigen must be present on the surface of the bacteria for this procedure to be effective, care was taken to ensure that ORF0657n was expressed on the bacteria prior to opsonization. The challenge strain was S. aureus RN4220, which was passaged 2× in the low iron medium RPMI. The dose of opsonized bacteria injected into each mouse was 1−2×10⁹ CFU/mouse. Results are shown in Table 13.

TABLE 13

| Monoclonal | #tests | Aggregate | % survival |
|---|---|---|---|
| 2H2.IgG1 | 6 | 30/30 | 100% |
| 10B4.IgG1 | 6 | 2/30 | 7% |
| Isotype control | | | |
| 13C7.IgG2b | 2 | 0/10 | 0% |
| 6G6.IgG2b | 2 | 0/10 | 0% |
| Isotype control | | | |
| CS-D7 | 4 | 5/20 | 25% |
| 20C2HA | 4 | 3/20 | 15% |
| Isotype control | | | |

### Example 11

### Opsonophagocytic Assay

An opsonophagocytosis activity (OPA) assay was developed to evaluate an antibody's ability to opsonophagocytose. The assay measures the ability of antibodies to bind and fix complement (C′) to the bacterial surface, which results in an increase in the level of phagocytosis of these bacteria by granulocytic effecter cells.

ORF0657n is an iron regulated protein on the surface of S. aureus that appears to be involved in heme/Fe acquisition. The S. aureus strain used in this assay is a strain that does not make protein A. An example of such a strain is S. aureus SH1000. For this assay the strain is iron starved to increase the expression of ORF0657n. This strain also lacks the ability to produce Protein A. Protein A can bind to the Fc portion of any IgG and the presence of this non-specifically bound antibody could interfere in the OPA reaction.

HL60 cells were exposed to dimethylformamide (DMF) for five to six days to induce the cells to differentiate towards a more granulocytic phenotype. Next, 2% C′-sufficient gnotobiotic pig serum was added to the antibody bound cells. Finally, the antibody and C′ exposed cells were then labeled with the fluorescent chemical 5′, 6′-FAM-SE.

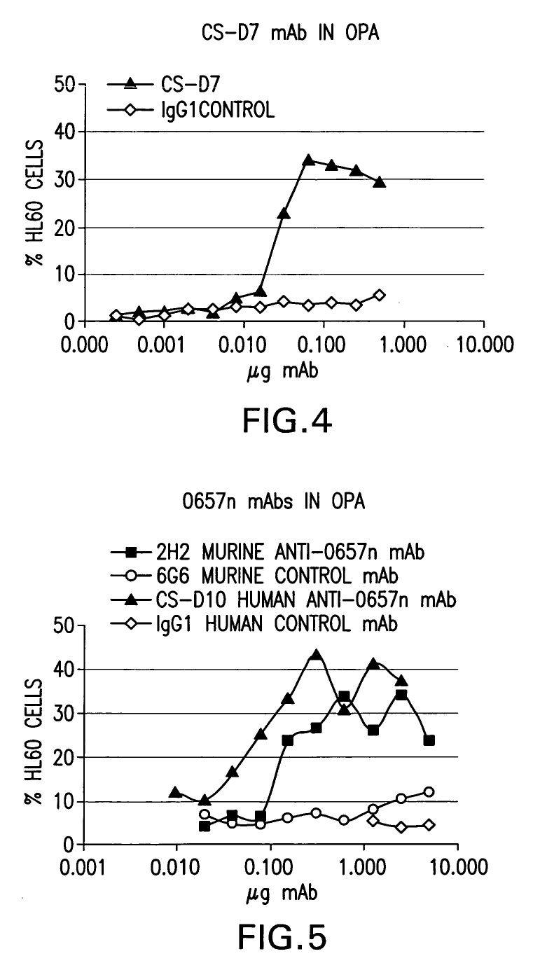

After incubation of opsonized, fluorescently-labeled bacteria and unlabeled HL60_DMF cells, the level of phagocytosis was determined by measuring the percentage of HL60_DMF cells containing labeled bacteria by flow cytometry. The percentage of HL60 cells with engulfed bacteria is proportional to the amount of opsinization induced by the antibody. Both murine and human mAbs to ORF0657n were examined in this assay. The results are illustrated in FIGS. 4 and 5.

The ORF0657n-specific mAbs were able to produce titratable activity in this assay. The murine mAb 2H2.BE11 had greater activity than the murine isotype control mAb, 6G6.A8. The human mAb CS-D7 and mAb CS-D10 also had higher opsonic activity compared to their IgG1 isotype control. The quantity of mAb needed to produce a maximal level of phagocytosis ranged from 0.5 ug for murine mAb 2H2.BE11 to 0.06-0.25 ug for the human mAb. The human mAb CS-D7 only needed 0.06 ug to generate a maximum level of phagocytosis as compared to 0.5 ug of murine mAb 2H2.BE11.

### Example 12

### Additional Sequences

SEQ ID NO: 47, which provides a S. aureus ORF0657n sequence, is as follows:

```
Met Asn Lys Gln Gln Lys Glu Phe Lys Ser Phe Tyr Ser Ile Arg Lys
  1 5 10 15
Ser Ser Leu Gly Val Ala Ser Val Ala Ile Ser Thr Leu Leu Leu Leu
                        20 25 30
Met Ser Asn Gly Glu Ala Gln Ala Ala Ala Glu Glu Thr Gly Gly Thr
                35 40 45
Asn Thr Glu Ala Gln Pro Lys Thr Glu Ala Val Ala Ser Pro Thr Thr
        50 55 60
Thr Ser Glu Lys Ala Pro Glu Thr Lys Pro Val Ala Asn Ala Val Ser
65 70 75 80
Val Ser Asn Lys Glu Val Glu Ala Pro Thr Ser Glu Thr Lys Glu Ala
                                85 90 95
Lys Glu Val Lys Glu Val Lys Ala Pro Lys Glu Thr Lys Ala Val Lys
                        100 105 110
Pro Ala Ala Lys Ala Thr Asn Asn Thr Tyr Pro Ile Leu Asn Gln Glu
                115 120 125
Leu Arg Glu Ala Ile Lys Asn Pro Ala Ile Lys Asp Lys Asp His Ser
        130 135 140
Ala Pro Asn Ser Arg Pro Ile Asp Phe Glu Met Lys Lys Glu Asn Gly
145 150 155 160
Glu Gln Gln Phe Tyr His Tyr Ala Ser Ser Val Lys Pro Ala Arg Val
                                165 170 175
Ile Phe Thr Asp Ser Lys Pro Glu Ile Glu Leu Gly Leu Gln Ser Gly
                        180 185 190
Gln Phe Trp Arg Lys Phe Glu Val Tyr Glu Gly Asp Lys Lys Leu Pro
                195 200 205
Ile Lys Leu Val Ser Tyr Asp Thr Val Lys Asp Tyr Ala Tyr Ile Arg
        210 215 220
Phe Ser Val Ser Asn Gly Thr Lys Ala Val Lys Ile Val Ser Ser Thr
225 230 235 240
His Phe Asn Asn Lys Glu Glu Lys Tyr Asp Tyr Thr Leu Met Glu Phe
                                245 250 255
Ala Gln Pro Ile Tyr Asn Ser Ala Asp Lys Phe Lys Thr Glu Glu Asp
                        260 265 270
Tyr Lys Ala Glu Lys Leu Leu Ala Pro Tyr Lys Lys Ala Lys Thr Leu
                275 280 285
Glu Arg Gln Val Tyr Glu Leu Asn Lys Ile Gln Asp Lys Leu Pro Glu
        290 295 300
Lys Leu Lys Ala Glu Tyr Lys Lys Lys Leu Glu Asp Thr Lys Lys Ala
305 310 315 320
Leu Asp Glu Gln Val Lys Ser Ala Ile Thr Glu Phe Gln Asn Val Gln
                                325 330 335
Pro Thr Asn Glu Lys Met Thr Asp Leu Gln Asp Thr Lys Tyr Val Val
                        340 345 350
Tyr Glu Ser Val Glu Asn Asn Glu Ser Met Met Asp Thr Phe Val Lys
                355 360 365
His Pro Ile Lys Thr Gly Met Leu Asn Gly Lys Lys Tyr Met Val Met
        370 375 380
Glu Thr Thr Asn Asp Asp Tyr Trp Lys Asp Phe Met Val Glu Gly Gln
385 390 395 400
Arg Val Arg Thr Ile Ser Lys Asp Ala Lys Asn Asn Thr Arg Thr Ile
                                405 410 415
Ile Phe Pro Tyr Val Glu Gly Lys Thr Leu Tyr Asp Ala Ile Val Lys
                        420 425 430
Val His Val Lys Thr Ile Asp Tyr Asp Gly Gln Tyr His Val Arg Ile
                435 440 445
Val Asp Lys Glu Ala Phe Thr Lys Ala Asn Thr Asp Lys Ser Asn Lys
        450 455 460
Lys Glu Gln Gln Asp Asn Ser Ala Lys Lys Glu Ala Thr Pro Ala Thr
465 470 475 480
Pro Ser Lys Pro Thr Pro Ser Pro Val Glu Lys Glu Ser Gln Lys Gln
                                485 490 495
Asp Ser Gln Lys Asp Asp Asn Lys Gln Leu Pro Ser Val Glu Lys Glu
                        500 505 510
Asn Asp Ala Ser Ser Glu Ser Gly Lys Asp Lys Thr Pro Ala Thr Lys
                515 520 525
Pro Thr Lys Gly Glu Val Glu Ser Ser Ser Thr Thr Pro Thr Lys Val
        530 535 540
Val Ser Thr Thr Gln Asn Val Ala Lys Pro Thr Thr Ala Ser Ser Lys
545 550 555 560
Thr Thr Lys Asp Val Val Gln Thr Ser Ala Gly Ser Ser Glu Ala Lys
                                565 570 575
Asp Ser Ala Pro Leu Gln Lys Ala Asn Ile Lys Asn Thr Asn Asp Gly
                        580 585 590
His Thr Gln Ser Gln Asn Asn Lys Asn Thr Gln Glu Asn Lys Ala Lys
                595 600 605
Ser Leu Pro Gln Thr Gly Glu Glu Ser Asn Lys Asp Met Thr Leu Pro
        610 615 620
Leu Met Ala Leu Leu Ala Leu Ser Ser Ile Val Ala Phe Val Leu Pro
625 630 635 640
Arg Lys Arg Lys Asn
                                645
```

SEQ ID NO: 48, which provides a human lambda sequence, is as follows:

```
Gln Pro Lys Ala Asn Pro Thr Val Thr Leu Phe Pro Pro Ser Ser Glu
  1 5 10 15
Glu Leu Gln Ala Asn Lys Ala Thr Leu Val Cys Leu Ile Ser Asp Phe
                        20 25 30
Tyr Pro Gly Ala Val Thr Val Ala Trp Lys Ala Asp Gly Ser Pro Val
                35 40 45
Lys Ala Gly Val Glu Thr Thr Lys Pro Ser Lys Gln Ser Asn Asn Lys
        50 55 60
Tyr Ala Ala Ser Ser Tyr Leu Ser Leu Thr Pro Glu Gln Trp Lys Ser
65 70 75 80
His Arg Ser Tyr Ser Cys Gln Val Thr His Glu Gly Ser Thr Val Glu
                                85 90 95
Lys Thr Val Ala Pro Thr Glu Cys Ser
                        100 105
```

### Example 13

### Therapeutic Administration of mAb CS-D7

Cannulated rats were challenged with 1−2×10⁹ CFU S. aureus via the tail vein. After 1 hour, rats were injected ip with either 4 mg monoclonal antibody (mAb CS-D7 or an isotype contro) or PBS alone. At 24 hours post challenge, the rats were sacrificed and the catheters removed. Catheters were evaluated for S. aureus by plating on mannitol salt plates overnight. The results of four different experiments are shown in Table 14.

TABLE 14
Rat Indwelling Catheter Model, Therapeutic Administration

| | Number of Culture - Negative Catheters | | | | | |
|---|---|---|---|---|---|---|
| mAb (quantity) | Exp#1 | Exp#2 | Exp#3 | Exp#4 | Total | p value |
| CS-D7 (4 mg) | 2 of 3 (60%) | 0 of 5 (0%) | 4 of 5 (80%) | 7 of 10 (70%) | 13 of 23 (56%) | 0.0059* |
| 20C2HA (4 mg) | 0 of 3 (0%) | 0 of 5 (0%) | 1 of 5 (20%) | 4 of 10 (40%) | 5 of 23 (22%) | |
| (isotype control) | | | | | | |
| PBS | nd | nd | 0 of 5 (0%) | 0 of 10 (0%) | 0 of 15 (0%) | <0.0001** |

*p value comparing mAb CS-D7 to mAb 20C2HA
**p value comparing mAb CS-D7 to PBS

### Example 14

### Combination Treatment—Vancomycin and mAb CS-D7

Cannulated rats were given vancomycin (20 mpk) sub. cu. at 1 hour (1 H) prior to iv challenge with 2−4×10⁹ CFU of S. aureus Becker. At 1 H post challenge, the rats were injected with 6 mg/rat of either the mAb CS-D6, an isotype control mAb 20C2HA, or PBS. At 24 hours post challenge, the animals were sacrificed, the jugular vein catheters harvested and evaluated for the bacteria load on the catheter. Evaluation of catheters was performed by placement in selective broth medium, and outgrowth on the Piccolo incubation system. Outgrowth was compared to standard curves of S. aureus growth under the same conditions to calculate the number of CFU on the experimental catheters. In the first set of experiments (Table 15), small numbers of animals were used to test the system. In the second set of experiments (Table 16), larger numbers of rats were used. In both cases, there was a significant enhancement of the activity of vancomycin in the presence of mAb CS-D7 than in the presence of the isotype control mAb. This indicates that mAb CS-D7 can enhance bacterial clearance above vancomycin alone. This model was designed to simulate a clinical situation in which a patient is undergoing surgery or other invasive procedure, and getting an empiric or prophylactic antibiotic. In the model, the mAb was injected after bacteria exposure, simulating adjunctive mAb treatment for infection during surgery. Under these very stringent conditions, the mAb had a beneficial effect.

TABLE 15
mAb CS-D7 enhances anti-Staph activity of vancomycin versus
isotype control mAb, reducing catheter colonization of cannulated
rats challenged with S. aureus (Becker).

| | | Catheter CFU^{@} | Catheter CFU | |
|---|---|---|---|---|
| mAb group | | Exp#1 | Exp#2 | Geo mean |
| 1. | Vancomycin + PBS | 0 | 0 | 45 |
| | | 38,725,264 | 217 | |
| | | | 0 | |
| | | | 0 | |
| 2. | Vancomycin + | 0 | 22 | 34* |
| | mAb CS-D7 | 0 | 963 | |
| | | | 115 | |
| | | | 678 | |
| 3. | Vancomycin + Isotype | 0 | 0 | 4,235 |
| | control mAb | 38,725,264 | 10,119 | |
| | | | 5,008 | |
| | | | 2,941,261 | |

^{@}Catheters with no outgrowth assigned value of “1” for geo mean determination
*p = 0.035 for group 2 vs group 3

TABLE 16
mAb CS-D7 enhances anti-Staph activity of vancomycin versus
isotype control mAb, reducing catheter colonization of cannulated
rats challenged with S. aureus (Becker).

| | | Catheter CFU^{@} | Catheter CFU | |
|---|---|---|---|---|
| mAb group | | Exp#1 | Exp#2 | Geo mean |
| 1. | PBS alone | 0 | 0 | 437,814 |
| | | 2,733 | 153,403 | |
| | | 26,707,702 | 5,555,250 | |
| | | 35,346,748 | 12,130,397 | |
| | | 47,050,905 | 16,544,368 | |
| | | 54,567,546 | | |
| 2. | Vancomycin + | 64 | 0 | 3,368 |
| | PBS | 309 | 63 | |
| | | 973 | 63 | |
| | | 5,015 | 291 | |
| | | 5,108 | 6,517,926 | |
| | | 7,353,005 | 7,829,527 | |
| 3. | Vancomycin + | 0 | 0 | 127* |
| | mAb CS-D7 | 68 | 0 | |
| | | 217 | 0 | |
| | | 3,176 | 0 | |
| | | 8,113,017 | 63 | |
| | | | 63 | |
| 4. | Vancomycin + | 0 | 0 | 1,881 |
| | Isotype | 0 | 0 | |
| | control mAb | 618 | 0 | |
| | | 736,335 | 63 | |
| | | 930,866 | 85,395 | |
| | | 6,755,875 | 1,274,668 | |

^{@}Catheters with no outgrowth assigned value of “10” for geo mean determination
*p = 0.05 for group 3 vs group 4

Other embodiments are within the following claims. While several embodiments have been shown and described, various modifications may be made without departing from the spirit and scope of the present invention.

1. An isolated antigen binding protein comprising a first variable region and a second variable region, wherein said first and second variable region binds to a CS-D7 target region.

2. The binding protein of claim 1, wherein said first variable region is a heavy chain variable (Vₕ) region comprising at least one complementarity determining region (CDR) selected from the group consisting of:
a first Vₕ CDR comprising SEQ ID NO: 46 or a sequence differing from SEQ ID NO: 46 by one amino acid;
a second Vₕ CDR comprising either SEQ ID NO: 36, SEQ ID NO: 38, SEQ ID NO: 39,
SEQ ID NO: 41, SEQ ID NO: 43 or SEQ ID NO: 44, or a sequence differing from SEQ ID NOs: 36, 38, 39, 41, 43, or 44 by one amino acid; and,
a third Vₕ CDR comprising either SEQ ID NO: 37, SEQ ID NO: 42 or SEQ ID NO: 45, or a sequence differing from SEQ ID NOs: 37, 42, or 45 by one amino acid.

3. The binding protein of claim 2, wherein said Vₕ region comprises said first Vₕ CDR, said second Vₕ CDR and said third Vₕ CDR.

4. The binding protein of claim 3, wherein said first, second and third Vₕ CDRs, respectively, comprise the amino acid sequences selected from the group consisting of:
a) SEQ ID NO: 35, SEQ ID NO: 36 and SEQ ID NO: 37;
b) SEQ ID NO: 35, SEQ ID NO: 38 and SEQ ID NO: 37;
c) SEQ ID NO: 35, SEQ ID NO: 39 and SEQ ID NO: 37;
d) SEQ ID NO: 40, SEQ ID NO: 41 and SEQ ID NO: 42;
e) SEQ ID NO: 40, SEQ ID NO: 43 and SEQ ID NO: 45; and,
f) SEQ ID NO: 40, SEQ ID NO: 44 and SEQ ID NO: 42.

5. The binding protein of claim 2, wherein said second variable region is a light chain variable (Vₗ) region comprising at least one complementarity determining region (CDR) selected from the group consisting of:
a first Vₗ CDR comprising either SEQ ID NO: 17, SEQ ID NO: 20, SEQ ID NO: 23, SEQ ID NO: 26, SEQ ID NO: 29 or SEQ ID NO: 32, or a sequence differing from SEQ ID NOs: 17, 20, 23, 26, 29, or 32 by one amino acid;
a second Vₗ CDR comprising either SEQ ID NO: 18, SEQ ID NO: 21, SEQ ID NO: 24, SEQ ID NO: 27, SEQ ID NO: 30 or SEQ ID NO: 33, or a sequence differing from SEQ ID NOs: 18, 21, 24, 27, 30 or 33 by one amino acid; and,
a third Vₗ CDR comprising either SEQ ID NO: 19, SEQ ID NO: 22, SEQ ID NO: 25,
SEQ ID NO: 28, SEQ ID NO: 31 or SEQ ID NO: 34, or a sequence differing from SEQ ID NOs: 19, 22, 25, 28, 31, or 34 by one amino acid.

6. The binding protein of claim 5, wherein said Vₗ region comprises said first Vₗ CDR, said second Vₗ CDR and said third Vₗ CDR.

7. The binding protein of claim 6, wherein said first, second and third Vₗ CDRs, respectively, comprise the amino acid sequences selected from the group consisting of:
a) SEQ ID NO: 17, SEQ ID NO: 18 and SEQ ID NO: 19;
b) SEQ ID NO: 20, SEQ ID NO: 21 and SEQ ID NO: 22;
c) SEQ ID NO: 23, SEQ ID NO: 24 and SEQ ID NO: 25;
d) SEQ ID NO: 26, SEQ ID NO: 27 and SEQ ID NO: 28;
e) SEQ ID NO: 29, SEQ ID NO: 30 and SEQ ID NO: 31; and,
f) SEQ ID NO: 32, SEQ ID NO: 33 and SEQ ID NO: 34.

8. The binding protein of claim 7, wherein said binding protein is an antibody.

9. The binding protein of claim 8, wherein said first Vₕ CDR, said second Vₕ CDR and said third Vₕ CDR, respectively, comprise SEQ ID NO: 35, SEQ ID NO: 36 and SEQ ID NO: 37; and, said first Vₗ CDR, said second Vₗ CDR, and said third Vₗ CDR, respectively, comprise SEQ ID NO: 17, SEQ ID NO: 18 and SEQ ID NO: 19.

10. The binding protein of claim 8, wherein said Vₕ region comprises an amino acid sequence selected from the group consisting of amino acids 1-126 of SEQ ID NO: 2, SEQ ID NO: 4, SEQ ID NO: 6, SEQ ID NO: 8, SEQ ID NO: 10, SEQ ID NO: 12, SEQ ID NO: 14 and SEQ ID NO: 16.

11. The binding protein of claim 10, wherein said Vₗ region comprises an amino acid sequence selected from the group consisting of amino acids 1-108 of SEQ ID NO: 1, SEQ ID NO: 3, SEQ ID NO: 5, SEQ ID NO: 7, SEQ ID NO: 9, SEQ ID NO: 11, SEQ ID NO: 13 and SEQ ID NO: 15.

12. The binding protein of claim 8, wherein said antibody comprises either:
a) a Vₗ region comprising amino acids 1-108 of SEQ ID NO: 1 and a Vₕ region comprising amino acids 1-126 SEQ ID NO: 2;
b) a Vₗ region comprising SEQ ID NO: 3 and a Vₕ region comprising SEQ ID NO: 4;
c) a Vₗ region comprising SEQ ID NO: 5 and a Vₕ region comprising SEQ ID NO: 6;
d) a Vₗ region comprising SEQ ID NO: 7 and a Vₕ region comprising SEQ ID NO: 8;
e) a Vₗ region comprising SEQ ID NO: 9 and a Vₕ region comprising SEQ ID NO: 10;
f) a Vₗ region comprising SEQ ID NO: 11 and a Vₕ region comprising SEQ ID NO: 12;
g) a Vₗ region comprising SEQ ID NO: 13 and a Vₕ region comprising SEQ ID NO: 14;
Or,
h) Vₗ region comprising SEQ ID NO: 15 and a Vₕ region comprising SEQ ID NO: 16.

13. The binding protein of claim 12, wherein said Vₕ region comprises amino acids 1-126 of SEQ ID NO: 2 and said Vₗ region comprises amino acids 1-108 of SEQ ID NO: 1.

14. The binding protein of claim 8, wherein said antibody comprises a heavy chain comprising a hinge, CH₁, CH₂, and CH₃ regions from an IgG₁, IgG₂, IgG₃ or IgG₄ subtype; and a light chain comprising said Vₗ region, and either a human kappa Cₗ or human lambda Cₗ.

15. The binding protein of claim 1, wherein said binding protein is an antibody comprising a light chain which comprises SEQ ID NO: 1 and a heavy chain which comprises SEQ ID NO: 2.

16. A nucleic acid comprising at least one recombinant gene that encodes an antigen binding protein heavy chain variable (Vₕ) region or an antigen binding protein light chain variable (Vₗ) region as described in claim 5.

17. A nucleic acid of claim 16, wherein said nucleic acid comprises two recombinant genes, a first recombinant gene encoding the antigen binding protein Vₕ region and a second recombinant gene encoding the antigen binding protein Vₗ region.

18. A recombinant cell comprising the recombinant nucleic acid of claim 17.

19. A method of producing a protein comprising an antibody variable region comprising the steps of:
a) growing the recombinant cell of claim 18 under conditions wherein said protein is expressed; and,
b) purifying said protein.

20. A pharmaceutical composition comprising the binding protein of claim 1 and a pharmaceutically acceptable carrier.

21. A method of protecting or treating against an S. aureus infection in a patient comprising the step of administering to said patient an effective amount of the binding protein of claim 1.

22. The method of claim 21, wherein said patient is a human and said antigen binding protein is administered in conjunction with surgery or a foreign body implant.

23. The method of claim 21, wherein said patient is a human infected with S. aureus.

24. (canceled)

25. A polypeptide comprising an amino acid sequence with at least a 95% sequence identity to amino acids 42-342 of SEQ ID NO: 47, wherein said polypeptide is up to 350 amino acids in length.
